(19) United States
(12) Patent Application Publication
(10) Pub. No.: US 2021/0093399 A1
(43) Pub. Date: Apr. 1, 2021

(54) ROBOTICALLY-ACTUATED MEDICAL RETRACTORS

(71) Applicant: Auris Health, Inc., Redwood City, CA (US)

(72) Inventors: Alexander Tarek Hassan, San Francisco, CA (US); Travis Michael Schuh, Los Altos, CA (US)

(21) Appl. No.: 17/032,347

(22) Filed: Sep. 25, 2020

Related U.S. Application Data
(60) Provisional application No. 62/906,982, filed on Sep. 27, 2019.

(51) Int. Cl. A61B 34/30; A61B 34/00; A61B 17/02; A61B 90/50

(52) U.S. Cl. CPC A61B 34/30; A61B 34/71; A61B 2090/571; A61B 90/50; A61B 17/02

(57) ABSTRACT

The systems, devices, and methods of the present application relate to robotically-actuated liver retractors. A robotic medical system can include a first robotic arm, and a medical instrument comprising an instrument handle configured to attach to a distal end of a the robotic arm, a rigid proximal portion that extends from the instrument handle, and a distal portion configured to support a liver during a robotic medical procedure.

### PRIORITY AND RELATED APPLICATIONS

This application claims priority to U.S. Provisional Pat. App. No. 62/906,982, filed Sep. 27, 2019, which is incorporated herein by reference. Any and all applications for which a foreign or domestic priority claim is identified in the Application Data Sheet as filed with the present application are hereby incorporated by reference under 37 CFR 1.57.

### TECHNICAL FIELD

Systems and methods disclosed herein are related to robotic medical systems. More particularly, disclosed embodiments relate to robotically-actuated medical retractors configured for use with robotic medical systems.

### BACKGROUND

Medical procedures, such as laparoscopy, may involve accessing and visualizing the inside of a patient's anatomy for diagnostic and/or therapeutic purposes. Some laparoscopic procedures require liver retraction in order to obtain adequate exposure of the target anatomy. For example, liver retraction is common during cholecystectomy, Nissen fundoplication, gastric bypass, sleeve gastrectomy, and other procedures. During such procedures, liver retractors may be manually controlled by a bedside assistant and/or attached to the surgical table rail.

### BRIEF DESCRIPTION OF THE DRAWINGS

The disclosed aspects will hereinafter be described in conjunction with the appended drawings, provided to illustrate and not to limit the disclosed aspects, wherein like designations denote like elements.

FIG. 1 illustrates an embodiment of a cart-based robotic system arranged for diagnostic and/or therapeutic bronchoscopy.

FIG. 2 depicts further aspects of the robotic system of FIG. 1.

FIG. 3 illustrates an embodiment of the robotic system of FIG. 1 arranged for ureteroscopy.

FIG. 4 illustrates an embodiment of the robotic system of FIG. 1 arranged for a vascular procedure.

FIG. 5 illustrates an embodiment of a table-based robotic system arranged for a bronchoscopic procedure.

FIG. 6 provides an alternative view of the robotic system of FIG. 5.

FIG. 7 illustrates an example system configured to stow robotic arm(s).

FIG. 8 illustrates an embodiment of a table-based robotic system configured for a ureteroscopic procedure.

FIG. 9 illustrates an embodiment of a table-based robotic system configured for a laparoscopic procedure.

FIG. 10 illustrates an embodiment of the table-based robotic system of FIGS. 5-9 with pitch or tilt adjustment.

FIG. 11 provides a detailed illustration of the interface between the table and the column of the table-based robotic system of FIGS. 5-10.

FIG. 12 illustrates an alternative embodiment of a table-based robotic system.

FIG. 13 illustrates an end view of the table-based robotic system of FIG. 12.

FIG. 14 illustrates an end view of a table-based robotic system with robotic arms attached thereto.

FIG. 15 illustrates an exemplary instrument driver.

FIG. 16 illustrates an exemplary medical instrument with a paired instrument driver.

FIG. 17 illustrates an alternative design for an instrument driver and instrument where the axes of the drive units are parallel to the axis of the elongated shaft of the instrument.

FIG. 18 illustrates an instrument having an instrument-based insertion architecture.

FIG. 19 illustrates an exemplary controller.

FIG. 20 depicts a block diagram illustrating a localization system that estimates a location of one or more elements of the robotic systems of FIGS. 1-10, such as the location of the instrument of FIGS. 16-18, in accordance to an example embodiment.

FIG. 21 illustrates an example of a manual liver retractor during use.

FIG. 22A illustrates an embodiment of a robotically-actuated medical retractor in an unarticulated configuration.

FIG. 22B illustrates the robotically-actuated medical retractor of FIG. 22A in an articulated configuration.

FIG. 23 illustrates an embodiment of an instrument base of a robotically-actuated medical retractor including a plurality of drive inputs for actuating the medical retractor.

FIGS. 24A-24C illustrate an embodiment of an articulable distal portion of a robotically-actuated medical retractor. FIG. 24A illustrates the distal portion in an unarticulated configuration, FIG. 24B illustrates the distal portion in a first articulated configuration, and FIG. 24C illustrates the distal portion in an second articulated configuration.

FIGS. 25A and 25B illustrate an embodiment of a robotically-actuated medical retractor that includes an articulable distal portion with three independently articulating sections. FIG. 25A illustrates an embodiment of a base of the medical retractor, and FIG. 25B illustrates an embodiment of the articulable distal portion of the medical retractor in an articulated configuration.

FIGS. 26A and 26B illustrate embodiments of a robotically-actuated medical retractor that includes a rigid, fixed, or non-articulating distal portion. FIG. 26A illustrates a generally straight embodiment, while FIG. 26B illustrates a bent embodiment.

FIG. 27A illustrates an embodiment a proximal portion of a robotically-actuated medical retractor that includes a connector at a distal end thereof, wherein the connector is configured to selectively attach to a distal portion of the medical retractor.

FIG. 27B illustrates an embodiment of an articulable distal portion of a medical retractor including a connector for connecting to a proximal portion of a medical retractor, such as the proximal portion of the medical retractor of FIG. 27A.

FIG. 27C illustrates an embodiment of a rigid, fixed, or non-articulating distal portion of a medical retractor including a connector for connecting to a proximal portion of a medical retractor, such as the proximal portion of the medical retractor of FIG. 27A.

FIG. 28 illustrates an embodiment of a robotic medical system including a robotic arm, an instrument comprising articulating segments, and a medical retractor.

FIG. 29 is a flowchart illustrating an example method for a robotically-enabled medical procedure using a robotically-actuated medical retractor.

### DETAILED DESCRIPTION

### 1. Overview

Aspects of the present disclosure may be integrated into a robotically-enabled medical system capable of performing a variety of medical procedures, including both minimally invasive, such as laparoscopy, and non-invasive, such as endoscopy, procedures. Among endoscopic procedures, the system may be capable of performing bronchoscopy, ureteroscopy, gastroscopy, etc.

In addition to performing the breadth of procedures, the system may provide additional benefits, such as enhanced imaging and guidance to assist the physician. Additionally, the system may provide the physician with the ability to perform the procedure from an ergonomic position without the need for awkward arm motions and positions. Still further, the system may provide the physician with the ability to perform the procedure with improved ease of use such that one or more of the instruments of the system can be controlled by a single user.

Various embodiments will be described below in conjunction with the drawings for purposes of illustration. It should be appreciated that many other implementations of the disclosed concepts are possible, and various advantages can be achieved with the disclosed implementations. Headings are included herein for reference and to aid in locating various sections. These headings are not intended to limit the scope of the concepts described with respect thereto. Such concepts may have applicability throughout the entire specification.

### A. Robotic System—Cart

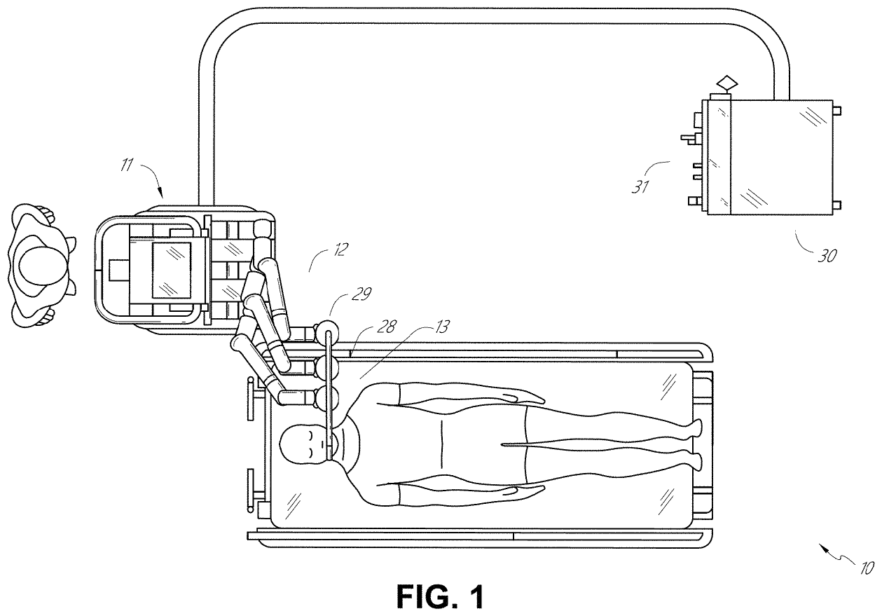

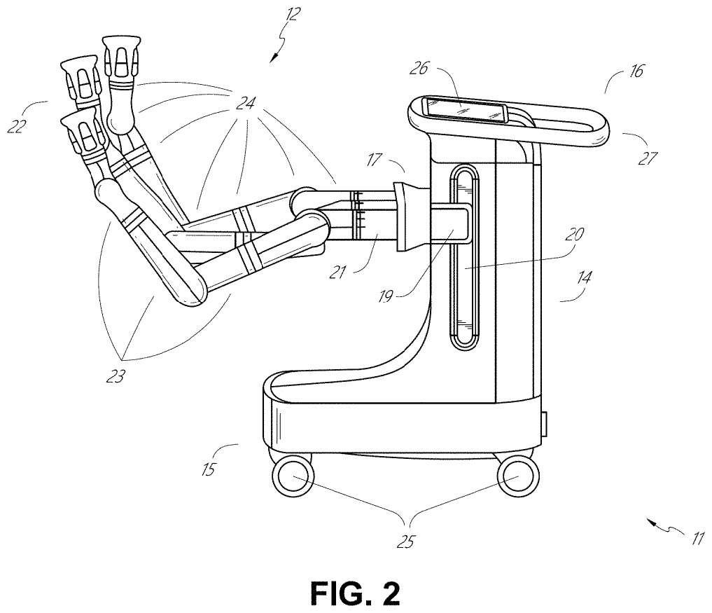

The robotically-enabled medical system may be configured in a variety of ways depending on the particular procedure. FIG. 1 illustrates an embodiment of a cart-based robotically-enabled system 10 arranged for a diagnostic and/or therapeutic bronchoscopy. During a bronchoscopy, the system 10 may comprise a cart 11 having one or more robotic arms 12 to deliver a medical instrument, such as a steerable endoscope 13, which may be a procedure-specific bronchoscope for bronchoscopy, to a natural orifice access point (i.e., the mouth of the patient positioned on a table in the present example) to deliver diagnostic and/or therapeutic tools. As shown, the cart 11 may be positioned proximate to the patient's upper torso in order to provide access to the access point. Similarly, the robotic arms 12 may be actuated to position the bronchoscope relative to the access point. The arrangement in FIG. 1 may also be utilized when performing a gastro-intestinal (GI) procedure with a gastroscope, a specialized endoscope for GI procedures. FIG. 2 depicts an example embodiment of the cart in greater detail.

With continued reference to FIG. 1, once the cart 11 is properly positioned, the robotic arms 12 may insert the steerable endoscope 13 into the patient robotically, manually, or a combination thereof. As shown, the steerable endoscope 13 may comprise at least two telescoping parts, such as an inner leader portion and an outer sheath portion, each portion coupled to a separate instrument driver from the set of instrument drivers 28, each instrument driver coupled to the distal end of an individual robotic arm. This linear arrangement of the instrument drivers 28, which facilitates coaxially aligning the leader portion with the sheath portion, creates a “virtual rail” 29 that may be repositioned in space by manipulating the one or more robotic arms 12 into different angles and/or positions. The virtual rails described herein are depicted in the Figures using dashed lines, and accordingly the dashed lines do not depict any physical structure of the system. Translation of the instrument drivers 28 along the virtual rail 29 telescopes the inner leader portion relative to the outer sheath portion or advances or retracts the endoscope 13 from the patient. The angle of the virtual rail 29 may be adjusted, translated, and pivoted based on clinical application or physician preference. For example, in bronchoscopy, the angle and position of the virtual rail 29 as shown represents a compromise between providing physician access to the endoscope 13 while minimizing friction that results from bending the endoscope 13 into the patient's mouth.

The endoscope 13 may be directed down the patient's trachea and lungs after insertion using precise commands from the robotic system until reaching the target destination or operative site. In order to enhance navigation through the patient's lung network and/or reach the desired target, the endoscope 13 may be manipulated to telescopically extend the inner leader portion from the outer sheath portion to obtain enhanced articulation and greater bend radius. The use of separate instrument drivers 28 also allows the leader portion and sheath portion to be driven independently of each other.

For example, the endoscope 13 may be directed to deliver a biopsy needle to a target, such as, for example, a lesion or nodule within the lungs of a patient. The needle may be deployed down a working channel that runs the length of the endoscope to obtain a tissue sample to be analyzed by a pathologist. Depending on the pathology results, additional tools may be deployed down the working channel of the endoscope for additional biopsies. After identifying a nodule to be malignant, the endoscope 13 may endoscopically deliver tools to resect the potentially cancerous tissue. In some instances, diagnostic and therapeutic treatments can be delivered in separate procedures. In those circumstances, the endoscope 13 may also be used to deliver a fiducial to “mark” the location of the target nodule as well. In other instances, diagnostic and therapeutic treatments may be delivered during the same procedure.

The system 10 may also include a movable tower 30, which may be connected via support cables to the cart 11 to provide support for controls, electronics, fluidics, optics, sensors, and/or power to the cart 11. Placing such functionality in the tower 30 allows for a smaller form factor cart 11 that may be more easily adjusted and/or re-positioned by an operating physician and his/her staff. Additionally, the division of functionality between the cart/table and the support tower 30 reduces operating room clutter and facilitates improving clinical workflow. While the cart 11 may be positioned close to the patient, the tower 30 may be stowed in a remote location to stay out of the way during a procedure.

In support of the robotic systems described above, the tower 30 may include component(s) of a computer-based control system that stores computer program instructions, for example, within a non-transitory computer-readable storage medium such as a persistent magnetic storage drive, solid state drive, etc. The execution of those instructions, whether the execution occurs in the tower 30 or the cart 11, may control the entire system or sub-system(s) thereof. For example, when executed by a processor of the computer system, the instructions may cause the components of the robotics system to actuate the relevant carriages and arm mounts, actuate the robotics arms, and control the medical instruments. For example, in response to receiving the control signal, the motors in the joints of the robotics arms may position the arms into a certain posture.

The tower 30 may also include a pump, flow meter, valve control, and/or fluid access in order to provide controlled irrigation and aspiration capabilities to the system that may be deployed through the endoscope 13. These components may also be controlled using the computer system of the tower 30. In some embodiments, irrigation and aspiration capabilities may be delivered directly to the endoscope 13 through separate cable(s).

The tower 30 may include a voltage and surge protector designed to provide filtered and protected electrical power to the cart 11, thereby avoiding placement of a power transformer and other auxiliary power components in the cart 11, resulting in a smaller, more moveable cart 11.

The tower 30 may also include support equipment for the sensors deployed throughout the robotic system 10. For example, the tower 30 may include optoelectronics equipment for detecting, receiving, and processing data received from the optical sensors or cameras throughout the robotic system 10. In combination with the control system, such optoelectronics equipment may be used to generate real-time images for display in any number of consoles deployed throughout the system, including in the tower 30. Similarly, the tower 30 may also include an electronic subsystem for receiving and processing signals received from deployed electromagnetic (EM) sensors. The tower 30 may also be used to house and position an EM field generator for detection by EM sensors in or on the medical instrument.

The tower 30 may also include a console 31 in addition to other consoles available in the rest of the system, e.g., console mounted on top of the cart. The console 31 may include a user interface and a display screen, such as a touchscreen, for the physician operator. Consoles in the system 10 are generally designed to provide both robotic controls as well as preoperative and real-time information of the procedure, such as navigational and localization information of the endoscope 13. When the console 31 is not the only console available to the physician, it may be used by a second operator, such as a nurse, to monitor the health or vitals of the patient and the operation of the system 10, as well as to provide procedure-specific data, such as navigational and localization information. In other embodiments, the console 30 is housed in a body that is separate from the tower 30.

The tower 30 may be coupled to the cart 11 and endoscope 13 through one or more cables or connections (not shown). In some embodiments, the support functionality from the tower 30 may be provided through a single cable to the cart 11, simplifying and de-cluttering the operating room. In other embodiments, specific functionality may be coupled in separate cabling and connections. For example, while power may be provided through a single power cable to the cart 11, the support for controls, optics, fluidics, and/or navigation may be provided through a separate cable.

FIG. 2 provides a detailed illustration of an embodiment of the cart 11 from the cart-based robotically-enabled system shown in FIG. 1. The cart 11 generally includes an elongated support structure 14 (often referred to as a “column”), a cart base 15, and a console 16 at the top of the column 14. The column 14 may include one or more carriages, such as a carriage 17 (alternatively “arm support”) for supporting the deployment of one or more robotic arms 12 (three shown in FIG. 2). The carriage 17 may include individually configurable arm mounts that rotate along a perpendicular axis to adjust the base of the robotic arms 12 for better positioning relative to the patient. The carriage 17 also includes a carriage interface 19 that allows the carriage 17 to vertically translate along the column 14.

The carriage interface 19 is connected to the column 14 through slots, such as slot 20, that are positioned on opposite sides of the column 14 to guide the vertical translation of the carriage 17. The slot 20 contains a vertical translation interface to position and hold the carriage 17 at various vertical heights relative to the cart base 15. Vertical translation of the carriage 17 allows the cart 11 to adjust the reach of the robotic arms 12 to meet a variety of table heights, patient sizes, and physician preferences. Similarly, the individually configurable arm mounts on the carriage 17 allow the robotic arm base 21 of the robotic arms 12 to be angled in a variety of configurations.

In some embodiments, the slot 20 may be supplemented with slot covers that are flush and parallel to the slot surface to prevent dirt and fluid ingress into the internal chambers of the column 14 and the vertical translation interface as the carriage 17 vertically translates. The slot covers may be deployed through pairs of spring spools positioned near the vertical top and bottom of the slot 20. The covers are coiled within the spools until deployed to extend and retract from their coiled state as the carriage 17 vertically translates up and down. The spring-loading of the spools provides force to retract the cover into a spool when the carriage 17 translates towards the spool, while also maintaining a tight seal when the carriage 17 translates away from the spool. The covers may be connected to the carriage 17 using, for example, brackets in the carriage interface 19 to ensure proper extension and retraction of the cover as the carriage 17 translates.

The column 14 may internally comprise mechanisms, such as gears and motors, that are designed to use a vertically aligned lead screw to translate the carriage 17 in a mechanized fashion in response to control signals generated in response to user inputs, e.g., inputs from the console 16.

The robotic arms 12 may generally comprise robotic arm bases 21 and end effectors 22, separated by a series of linkages 23 that are connected by a series of joints 24, each joint comprising an independent actuator, each actuator comprising an independently controllable motor. Each independently controllable joint represents an independent degree of freedom available to the robotic arm 12. Each of the robotic arms 12 may have seven joints, and thus provide seven degrees of freedom. A multitude of joints result in a multitude of degrees of freedom, allowing for “redundant” degrees of freedom. Having redundant degrees of freedom allows the robotic arms 12 to position their respective end effectors 22 at a specific position, orientation, and trajectory in space using different linkage positions and joint angles. This allows for the system to position and direct a medical instrument from a desired point in space while allowing the physician to move the arm joints into a clinically advantageous position away from the patient to create greater access, while avoiding arm collisions.

The cart base 15 balances the weight of the column 14, carriage 17, and robotic arms 12 over the floor. Accordingly, the cart base 15 houses heavier components, such as electronics, motors, power supply, as well as components that either enable movement and/or immobilize the cart 11. For example, the cart base 15 includes rollable wheel-shaped casters 25 that allow for the cart 11 to easily move around the room prior to a procedure. After reaching the appropriate position, the casters 25 may be immobilized using wheel locks to hold the cart 11 in place during the procedure.

Positioned at the vertical end of the column 14, the console 16 allows for both a user interface for receiving user input and a display screen (or a dual-purpose device such as, for example, a touchscreen 26) to provide the physician user with both preoperative and intraoperative data. Potential preoperative data on the touchscreen 26 may include preoperative plans, navigation and mapping data derived from preoperative computerized tomography (CT) scans, and/or notes from preoperative patient interviews. Intraoperative data on display may include optical information provided from the tool, sensor and coordinate information from sensors, as well as vital patient statistics, such as respiration, heart rate, and/or pulse. The console 16 may be positioned and tilted to allow a physician to access the console 16 from the side of the column 14 opposite the carriage 17. From this position, the physician may view the console 16, robotic arms 12, and patient while operating the console 16 from behind the cart 11. As shown, the console 16 also includes a handle 27 to assist with maneuvering and stabilizing the cart 11.

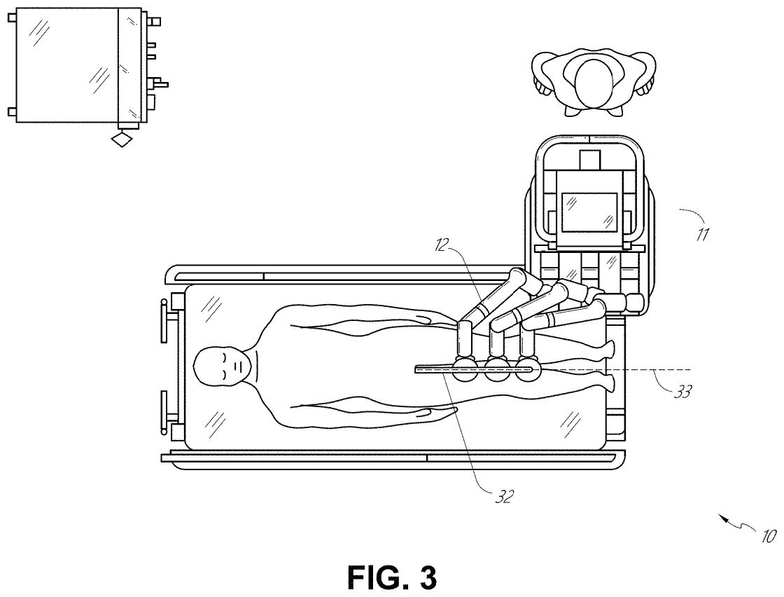

FIG. 3 illustrates an embodiment of a robotically-enabled system 10 arranged for ureteroscopy. In a ureteroscopic procedure, the cart 11 may be positioned to deliver a ureteroscope 32, a procedure-specific endoscope designed to traverse a patient's urethra and ureter, to the lower abdominal area of the patient. In a ureteroscopy, it may be desirable for the ureteroscope 32 to be directly aligned with the patient's urethra to reduce friction and forces on the sensitive anatomy in the area. As shown, the cart 11 may be aligned at the foot of the table to allow the robotic arms 12 to position the ureteroscope 32 for direct linear access to the patient's urethra. From the foot of the table, the robotic arms 12 may insert the ureteroscope 32 along the virtual rail 33 directly into the patient's lower abdomen through the urethra.

After insertion into the urethra, using similar control techniques as in bronchoscopy, the ureteroscope 32 may be navigated into the bladder, ureters, and/or kidneys for diagnostic and/or therapeutic applications. For example, the ureteroscope 32 may be directed into the ureter and kidneys to break up kidney stone build up using a laser or ultrasonic lithotripsy device deployed down the working channel of the ureteroscope 32. After lithotripsy is complete, the resulting stone fragments may be removed using baskets deployed down the ureteroscope 32.

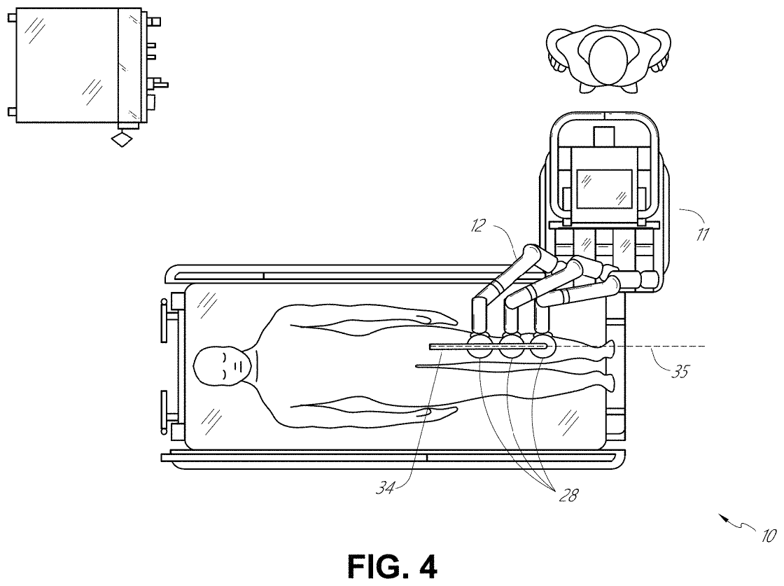

FIG. 4 illustrates an embodiment of a robotically-enabled system 10 similarly arranged for a vascular procedure. In a vascular procedure, the system 10 may be configured such that the cart 11 may deliver a medical instrument 34, such as a steerable catheter, to an access point in the femoral artery in the patient's leg. The femoral artery presents both a larger diameter for navigation as well as a relatively less circuitous and tortuous path to the patient's heart, which simplifies navigation. As in a ureteroscopic procedure, the cart 11 may be positioned towards the patient's legs and lower abdomen to allow the robotic arms 12 to provide a virtual rail 35 with direct linear access to the femoral artery access point in the patient's thigh/hip region. After insertion into the artery, the medical instrument 34 may be directed and inserted by translating the instrument drivers 28. Alternatively, the cart may be positioned around the patient's upper abdomen in order to reach alternative vascular access points, such as, for example, the carotid and brachial arteries near the shoulder and wrist.

### B. Robotic System—Table

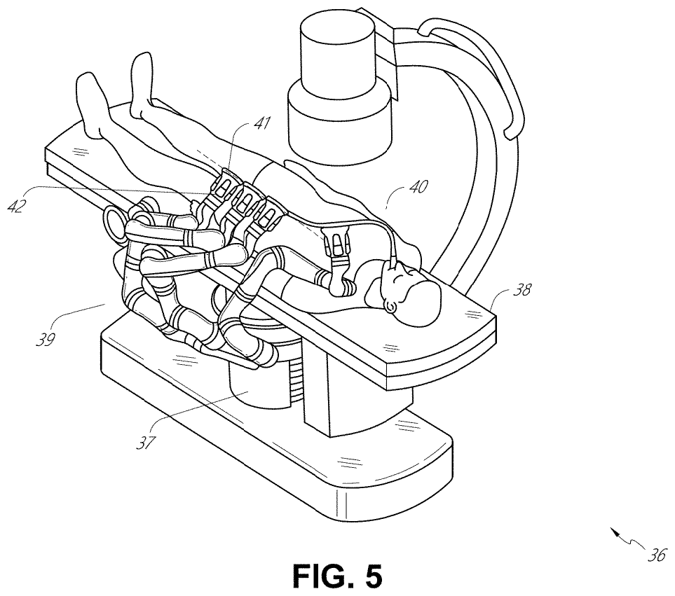

Embodiments of the robotically-enabled medical system may also incorporate the patient's table. Incorporation of the table reduces the amount of capital equipment within the operating room by removing the cart, which allows greater access to the patient. FIG. 5 illustrates an embodiment of such a robotically-enabled system arranged for a bronchoscopic procedure. System 36 includes a support structure or column 37 for supporting platform 38 (shown as a “table” or “bed”) over the floor. Much like in the cart-based systems, the end effectors of the robotic arms 39 of the system 36 comprise instrument drivers 42 that are designed to manipulate an elongated medical instrument, such as a bronchoscope 40 in FIG. 5, through or along a virtual rail 41 formed from the linear alignment of the instrument drivers 42. In practice, a C-arm for providing fluoroscopic imaging may be positioned over the patient's upper abdominal area by placing the emitter and detector around the table 38.

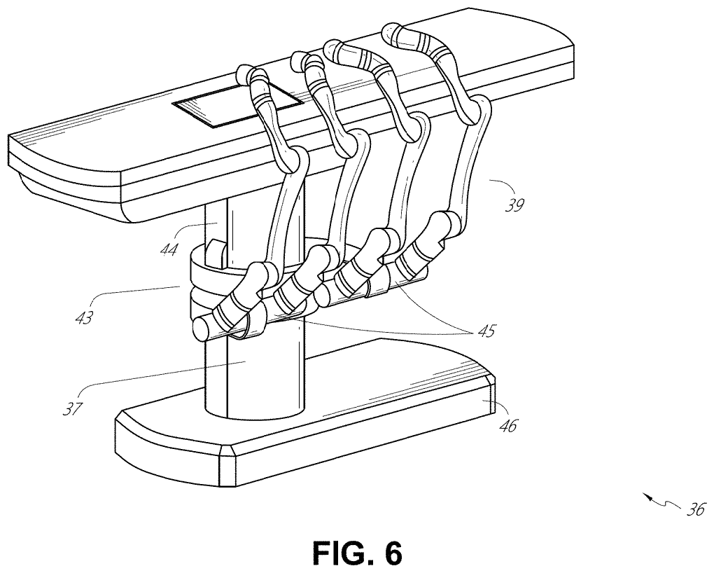

FIG. 6 provides an alternative view of the system 36 without the patient and medical instrument for discussion purposes. As shown, the column 37 may include one or more carriages 43 shown as ring-shaped in the system 36, from which the one or more robotic arms 39 may be based. The carriages 43 may translate along a vertical column interface 44 that runs the length of the column 37 to provide different vantage points from which the robotic arms 39 may be positioned to reach the patient. The carriage(s) 43 may rotate around the column 37 using a mechanical motor positioned within the column 37 to allow the robotic arms 39 to have access to multiples sides of the table 38, such as, for example, both sides of the patient. In embodiments with multiple carriages, the carriages may be individually positioned on the column and may translate and/or rotate independently of the other carriages. While the carriages 43 need not surround the column 37 or even be circular, the ring-shape as shown facilitates rotation of the carriages 43 around the column 37 while maintaining structural balance. Rotation and translation of the carriages 43 allows the system 36 to align the medical instruments, such as endoscopes and laparoscopes, into different access points on the patient. In other embodiments (not shown), the system 36 can include a patient table or bed with adjustable arm supports in the form of bars or rails extending alongside it. One or more robotic arms 39 (e.g., via a shoulder with an elbow joint) can be attached to the adjustable arm supports, which can be vertically adjusted. By providing vertical adjustment, the robotic arms 39 are advantageously capable of being stowed compactly beneath the patient table or bed, and subsequently raised during a procedure.

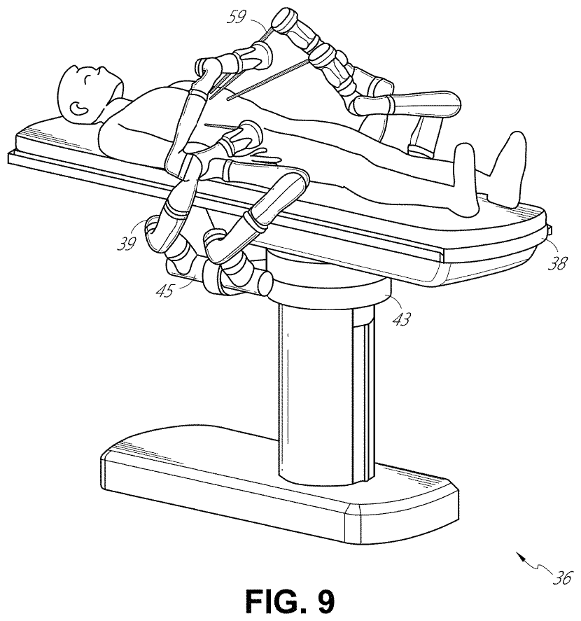

The robotic arms 39 may be mounted on the carriages 43 through a set of arm mounts 45 comprising a series of joints that may individually rotate and/or telescopically extend to provide additional configurability to the robotic arms 39. Additionally, the arm mounts 45 may be positioned on the carriages 43 such that, when the carriages 43 are appropriately rotated, the arm mounts 45 may be positioned on either the same side of the table 38 (as shown in FIG. 6), on opposite sides of the table 38 (as shown in FIG. 9), or on adjacent sides of the table 38 (not shown).

The column 37 structurally provides support for the table 38, and a path for vertical translation of the carriages 43. Internally, the column 37 may be equipped with lead screws for guiding vertical translation of the carriages, and motors to mechanize the translation of the carriages 43 based the lead screws. The column 37 may also convey power and control signals to the carriages 43 and the robotic arms 39 mounted thereon.

The table base 46 serves a similar function as the cart base 15 in the cart 11 shown in FIG. 2, housing heavier components to balance the table/bed 38, the column 37, the carriages 43, and the robotic arms 39. The table base 46 may also incorporate rigid casters to provide stability during procedures. Deployed from the bottom of the table base 46, the casters may extend in opposite directions on both sides of the base 46 and retract when the system 36 needs to be moved.

With continued reference to FIG. 6, the system 36 may also include a tower (not shown) that divides the functionality of the system 36 between the table and the tower to reduce the form factor and bulk of the table. As in earlier disclosed embodiments, the tower may provide a variety of support functionalities to the table, such as processing, computing, and control capabilities, power, fluidics, and/or optical and sensor processing. The tower may also be movable to be positioned away from the patient to improve physician access and de-clutter the operating room. Additionally, placing components in the tower allows for more storage space in the table base 46 for potential stowage of the robotic arms 39. The tower may also include a master controller or console that provides both a user interface for user input, such as keyboard and/or pendant, as well as a display screen (or touchscreen) for preoperative and intraoperative information, such as real-time imaging, navigation, and tracking information. In some embodiments, the tower may also contain holders for gas tanks to be used for insufflation.

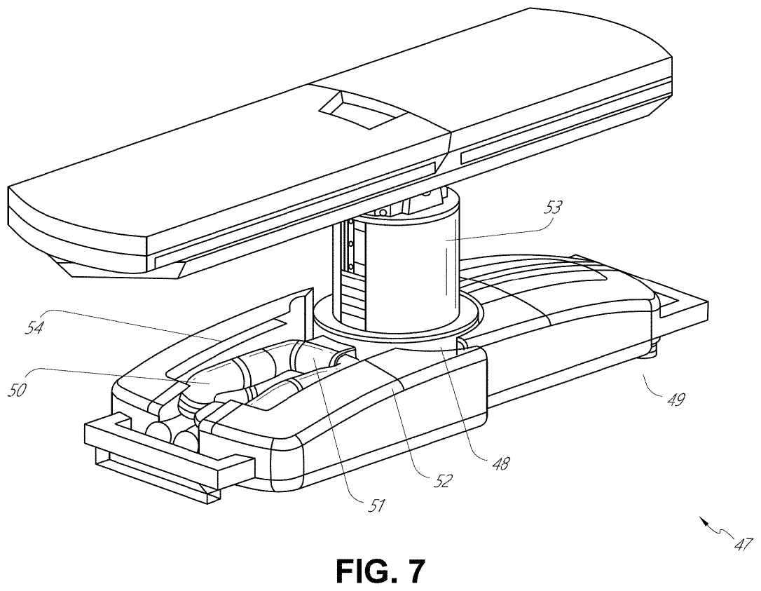

In some embodiments, a table base may stow and store the robotic arms when not in use. FIG. 7 illustrates a system 47 that stows robotic arms in an embodiment of the table-based system. In the system 47, carriages 48 may be vertically translated into base 49 to stow robotic arms 50, arm mounts 51, and the carriages 48 within the base 49. Base covers 52 may be translated and retracted open to deploy the carriages 48, arm mounts 51, and robotic arms 50 around column 53, and closed to stow to protect them when not in use. The base covers 52 may be sealed with a membrane 54 along the edges of its opening to prevent dirt and fluid ingress when closed.

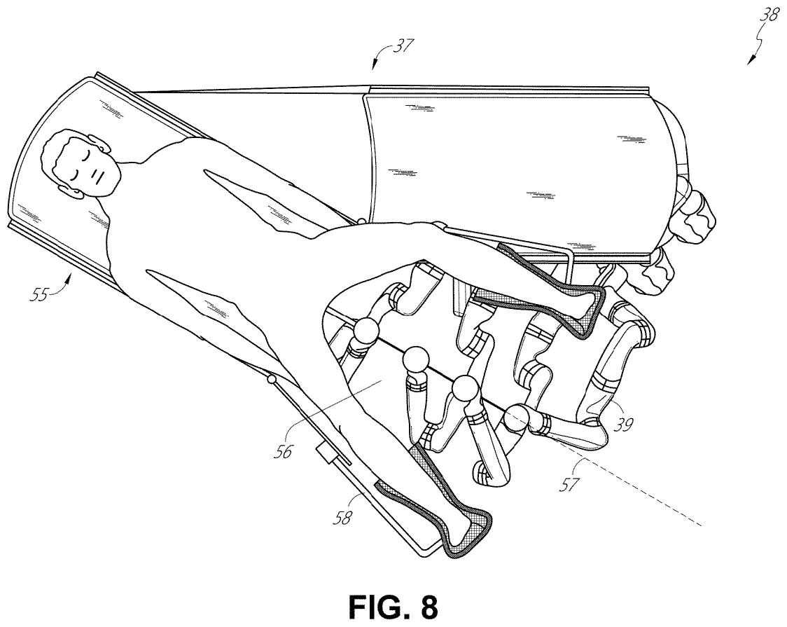

FIG. 8 illustrates an embodiment of a robotically-enabled table-based system configured for a ureteroscopic procedure. In a ureteroscopy, the table 38 may include a swivel portion 55 for positioning a patient off-angle from the column 37 and table base 46. The swivel portion 55 may rotate or pivot around a pivot point (e.g., located below the patient's head) in order to position the bottom portion of the swivel portion 55 away from the column 37. For example, the pivoting of the swivel portion 55 allows a C-arm (not shown) to be positioned over the patient's lower abdomen without competing for space with the column (not shown) below table 38. By rotating the carriage 35 (not shown) around the column 37, the robotic arms 39 may directly insert a ureteroscope 56 along a virtual rail 57 into the patient's groin area to reach the urethra. In a ureteroscopy, stirrups 58 may also be fixed to the swivel portion 55 of the table 38 to support the position of the patient's legs during the procedure and allow clear access to the patient's groin area.

In a laparoscopic procedure, through small incision(s) in the patient's abdominal wall, minimally invasive instruments may be inserted into the patient's anatomy. In some embodiments, the minimally invasive instruments comprise an elongated rigid member, such as a shaft, which is used to access anatomy within the patient. After inflation of the patient's abdominal cavity, the instruments may be directed to perform surgical or medical tasks, such as grasping, cutting, ablating, suturing, etc. In some embodiments, the instruments can comprise a scope, such as a laparoscope. FIG. 9 illustrates an embodiment of a robotically-enabled table-based system configured for a laparoscopic procedure. As shown in FIG. 9, the carriages 43 of the system 36 may be rotated and vertically adjusted to position pairs of the robotic arms 39 on opposite sides of the table 38, such that instrument 59 may be positioned using the arm mounts 45 to be passed through minimal incisions on both sides of the patient to reach his/her abdominal cavity.

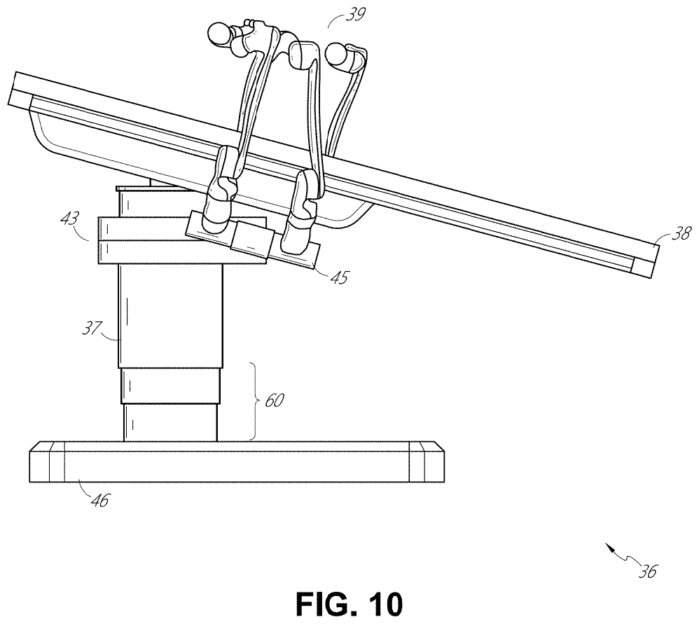

To accommodate laparoscopic procedures, the robotically-enabled table system may also tilt the platform to a desired angle. FIG. 10 illustrates an embodiment of the robotically-enabled medical system with pitch or tilt adjustment. As shown in FIG. 10, the system 36 may accommodate tilt of the table 38 to position one portion of the table at a greater distance from the floor than the other. Additionally, the arm mounts 45 may rotate to match the tilt such that the robotic arms 39 maintain the same planar relationship with the table 38. To accommodate steeper angles, the column 37 may also include telescoping portions 60 that allow vertical extension of the column 37 to keep the table 38 from touching the floor or colliding with the table base 46.

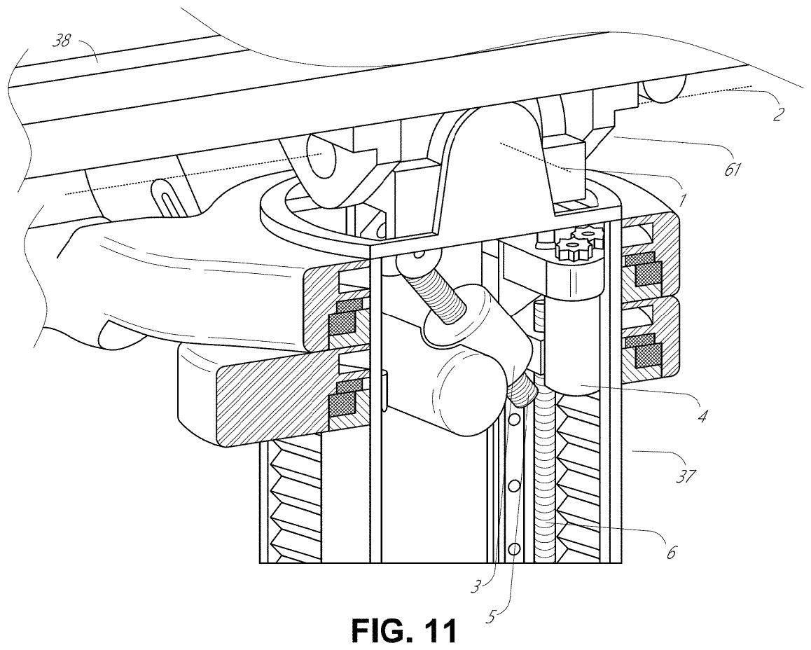

FIG. 11 provides a detailed illustration of the interface between the table 38 and the column 37. Pitch rotation mechanism 61 may be configured to alter the pitch angle of the table 38 relative to the column 37 in multiple degrees of freedom. The pitch rotation mechanism 61 may be enabled by the positioning of orthogonal axes 1, 2 at the column-table interface, each axis actuated by a separate motor 3, 4 responsive to an electrical pitch angle command. Rotation along one screw 5 would enable tilt adjustments in one axis 1, while rotation along the other screw 6 would enable tilt adjustments along the other axis 2. In some embodiments, a ball joint can be used to alter the pitch angle of the table 38 relative to the column 37 in multiple degrees of freedom.

For example, pitch adjustments are particularly useful when trying to position the table in a Trendelenburg position, i.e., position the patient's lower abdomen at a higher position from the floor than the patient's upper abdomen, for lower abdominal surgery. The Trendelenburg position causes the patient's internal organs to slide towards his/her upper abdomen through the force of gravity, clearing out the abdominal cavity for minimally invasive tools to enter and perform lower abdominal surgical or medical procedures, such as laparoscopic prostatectomy.

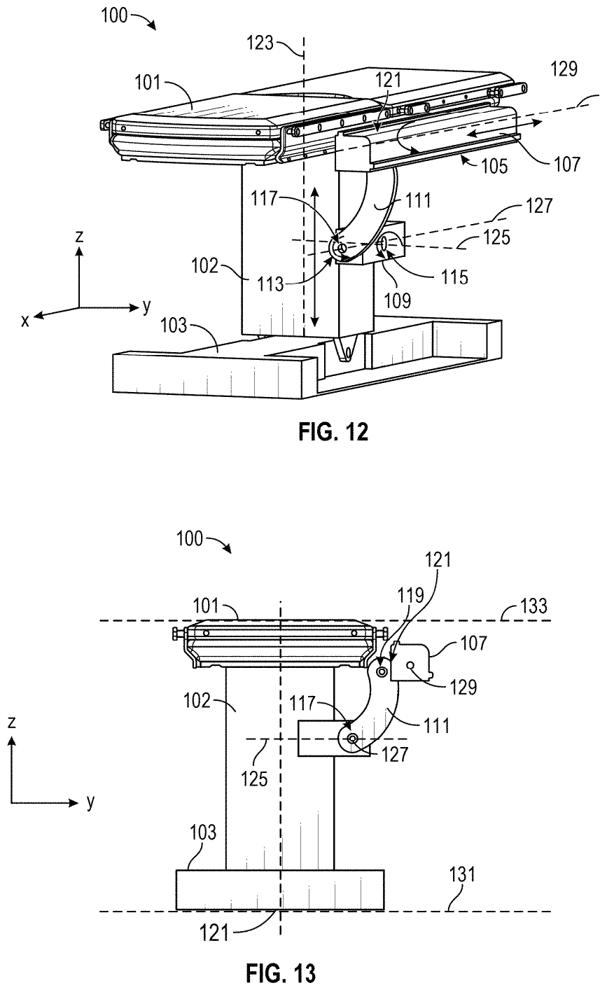

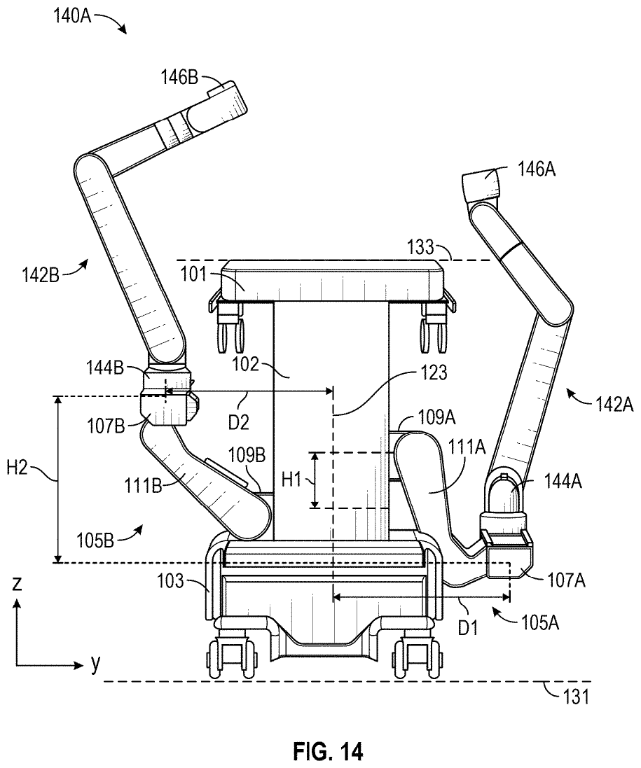

FIGS. 12 and 13 illustrate isometric and end views of an alternative embodiment of a table-based surgical robotics system 100. The surgical robotics system 100 includes one or more adjustable arm supports 105 that can be configured to support one or more robotic arms (see, for example, FIG. 14) relative to a table 101. In the illustrated embodiment, a single adjustable arm support 105 is shown, though an additional arm support can be provided on an opposite side of the table 101. The adjustable arm support 105 can be configured so that it can move relative to the table 101 to adjust and/or vary the position of the adjustable arm support 105 and/or any robotic arms mounted thereto relative to the table 101. For example, the adjustable arm support 105 may be adjusted one or more degrees of freedom relative to the table 101. The adjustable arm support 105 provides high versatility to the system 100, including the ability to easily stow the one or more adjustable arm supports 105 and any robotics arms attached thereto beneath the table 101. The adjustable arm support 105 can be elevated from the stowed position to a position below an upper surface of the table 101. In other embodiments, the adjustable arm support 105 can be elevated from the stowed position to a position above an upper surface of the table 101.

The adjustable arm support 105 can provide several degrees of freedom, including lift, lateral translation, tilt, etc. In the illustrated embodiment of FIGS. 12 and 13, the arm support 105 is configured with four degrees of freedom, which are illustrated with arrows in FIG. 12. A first degree of freedom allows for adjustment of the adjustable arm support 105 in the z-direction (“Z-lift”). For example, the adjustable arm support 105 can include a carriage 109 configured to move up or down along or relative to a column 102 supporting the table 101. A second degree of freedom can allow the adjustable arm support 105 to tilt. For example, the adjustable arm support 105 can include a rotary joint, which can allow the adjustable arm support 105 to be aligned with the bed in a Trendelenburg position. A third degree of freedom can allow the adjustable arm support 105 to “pivot up,” which can be used to adjust a distance between a side of the table 101 and the adjustable arm support 105. A fourth degree of freedom can permit translation of the adjustable arm support 105 along a longitudinal length of the table.

The surgical robotics system 100 in FIGS. 12 and 13 can comprise a table supported by a column 102 that is mounted to a base 103. The base 103 and the column 102 support the table 101 relative to a support surface. A floor axis 131 and a support axis 133 are shown in FIG. 13.

The adjustable arm support 105 can be mounted to the column 102. In other embodiments, the arm support 105 can be mounted to the table 101 or base 103. The adjustable arm support 105 can include a carriage 109, a bar or rail connector 111 and a bar or rail 107. In some embodiments, one or more robotic arms mounted to the rail 107 can translate and move relative to one another.

The carriage 109 can be attached to the column 102 by a first joint 113, which allows the carriage 109 to move relative to the column 102 (e.g., such as up and down a first or vertical axis 123). The first joint 113 can provide the first degree of freedom (“Z-lift”) to the adjustable arm support 105. The adjustable arm support 105 can include a second joint 115, which provides the second degree of freedom (tilt) for the adjustable arm support 105. The adjustable arm support 105 can include a third joint 117, which can provide the third degree of freedom (“pivot up”) for the adjustable arm support 105. An additional joint 119 (shown in FIG. 13) can be provided that mechanically constrains the third joint 117 to maintain an orientation of the rail 107 as the rail connector 111 is rotated about a third axis 127. The adjustable arm support 105 can include a fourth joint 121, which can provide a fourth degree of freedom (translation) for the adjustable arm support 105 along a fourth axis 129.

FIG. 14 illustrates an end view of the surgical robotics system 140A with two adjustable arm supports 105A, 105B mounted on opposite sides of a table 101. A first robotic arm 142A is attached to the bar or rail 107A of the first adjustable arm support 105B. The first robotic arm 142A includes a base 144A attached to the rail 107A. The distal end of the first robotic arm 142A includes an instrument drive mechanism 146A that can attach to one or more robotic medical instruments or tools. Similarly, the second robotic arm 142B includes a base 144B attached to the rail 107B. The distal end of the second robotic arm 142B includes an instrument drive mechanism 146B. The instrument drive mechanism 146B can be configured to attach to one or more robotic medical instruments or tools.

In some embodiments, one or more of the robotic arms 142A, 142B comprises an arm with seven or more degrees of freedom. In some embodiments, one or more of the robotic arms 142A, 142B can include eight degrees of freedom, including an insertion axis (1-degree of freedom including insertion), a wrist (3-degrees of freedom including wrist pitch, yaw and roll), an elbow (1-degree of freedom including elbow pitch), a shoulder (2-degrees of freedom including shoulder pitch and yaw), and base 144A, 144B (1-degree of freedom including translation). In some embodiments, the insertion degree of freedom can be provided by the robotic arm 142A, 142B, while in other embodiments, the instrument itself provides insertion via an instrument-based insertion architecture.

### C. Instrument Driver & Interface

The end effectors of the system's robotic arms may comprise (i) an instrument driver (alternatively referred to as “instrument drive mechanism” or “instrument device manipulator”) that incorporates electro-mechanical means for actuating the medical instrument and (ii) a removable or detachable medical instrument, which may be devoid of any electro-mechanical components, such as motors. This dichotomy may be driven by the need to sterilize medical instruments used in medical procedures, and the inability to adequately sterilize expensive capital equipment due to their intricate mechanical assemblies and sensitive electronics. Accordingly, the medical instruments may be designed to be detached, removed, and interchanged from the instrument driver (and thus the system) for individual sterilization or disposal by the physician or the physician's staff. In contrast, the instrument drivers need not be changed or sterilized, and may be draped for protection.

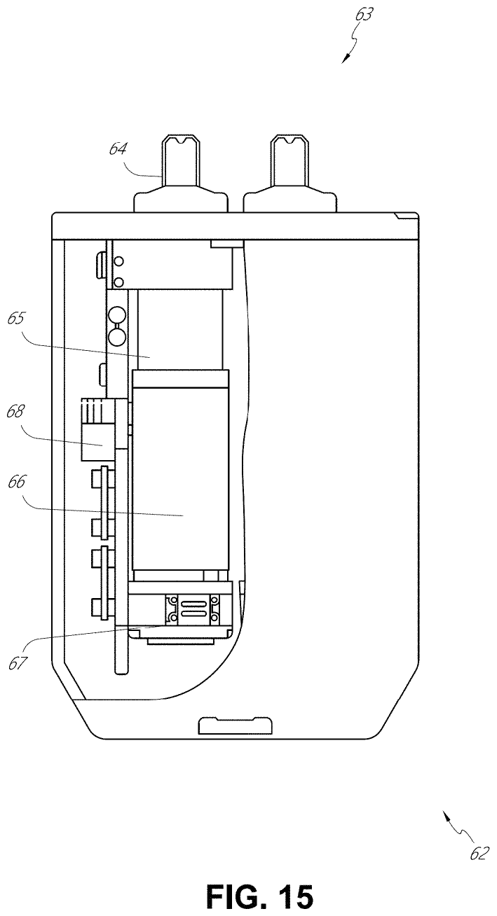

FIG. 15 illustrates an example instrument driver. Positioned at the distal end of a robotic arm, instrument driver 62 comprises one or more drive units 63 arranged with parallel axes to provide controlled torque to a medical instrument via drive shafts 64. Each drive unit 63 comprises an individual drive shaft 64 for interacting with the instrument, a gear head 65 for converting the motor shaft rotation to a desired torque, a motor 66 for generating the drive torque, an encoder 67 to measure the speed of the motor shaft and provide feedback to the control circuitry, and control circuity 68 for receiving control signals and actuating the drive unit. Each drive unit 63 being independently controlled and motorized, the instrument driver 62 may provide multiple (e.g., four as shown in FIG. 15) independent drive outputs to the medical instrument. In operation, the control circuitry 68 would receive a control signal, transmit a motor signal to the motor 66, compare the resulting motor speed as measured by the encoder 67 with the desired speed, and modulate the motor signal to generate the desired torque.

For procedures that require a sterile environment, the robotic system may incorporate a drive interface, such as a sterile adapter connected to a sterile drape, that sits between the instrument driver and the medical instrument. The chief purpose of the sterile adapter is to transfer angular motion from the drive shafts of the instrument driver to the drive inputs of the instrument while maintaining physical separation, and thus sterility, between the drive shafts and drive inputs. Accordingly, an example sterile adapter may comprise a series of rotational inputs and outputs intended to be mated with the drive shafts of the instrument driver and drive inputs on the instrument. Connected to the sterile adapter, the sterile drape, comprised of a thin, flexible material such as transparent or translucent plastic, is designed to cover the capital equipment, such as the instrument driver, robotic arm, and cart (in a cart-based system) or table (in a table-based system). Use of the drape would allow the capital equipment to be positioned proximate to the patient while still being located in an area not requiring sterilization (i.e., non-sterile field). On the other side of the sterile drape, the medical instrument may interface with the patient in an area requiring sterilization (i.e., sterile field).

### D. Medical Instrument

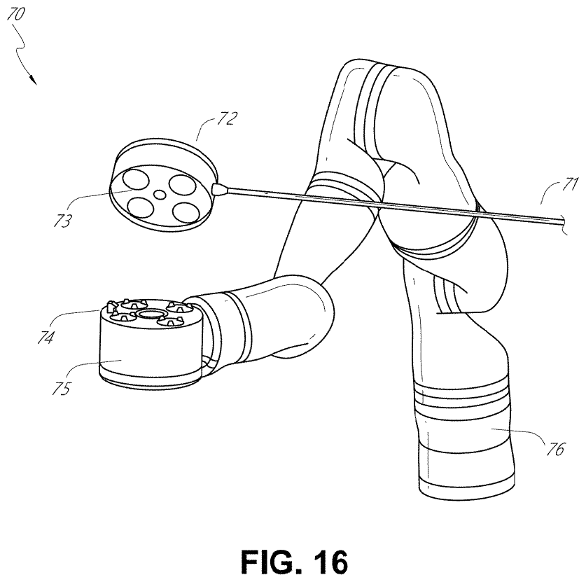

FIG. 16 illustrates an example medical instrument with a paired instrument driver. Like other instruments designed for use with a robotic system, medical instrument 70 comprises an elongated shaft 71 (or elongate body) and an instrument base 72. The instrument base 72, also referred to as an “instrument handle” due to its intended design for manual interaction by the physician, may generally comprise rotatable drive inputs 73, e.g., receptacles, pulleys or spools, that are designed to be mated with drive outputs 74 that extend through a drive interface on instrument driver 75 at the distal end of robotic arm 76. When physically connected, latched, and/or coupled, the mated drive inputs 73 of the instrument base 72 may share axes of rotation with the drive outputs 74 in the instrument driver 75 to allow the transfer of torque from the drive outputs 74 to the drive inputs 73. In some embodiments, the drive outputs 74 may comprise splines that are designed to mate with receptacles on the drive inputs 73.

The elongated shaft 71 is designed to be delivered through either an anatomical opening or lumen, e.g., as in endoscopy, or a minimally invasive incision, e.g., as in laparoscopy. The elongated shaft 71 may be either flexible (e.g., having properties similar to an endoscope) or rigid (e.g., having properties similar to a laparoscope) or contain a customized combination of both flexible and rigid portions. When designed for laparoscopy, the distal end of a rigid elongated shaft may be connected to an end effector extending from a jointed wrist formed from a clevis with at least one degree of freedom and a surgical tool or medical instrument, such as, for example, a grasper or scissors, that may be actuated based on force from the tendons as the drive inputs rotate in response to torque received from the drive outputs 74 of the instrument driver 75. When designed for endoscopy, the distal end of a flexible elongated shaft may include a steerable or controllable bending section that may be articulated and bent based on torque received from the drive outputs 74 of the instrument driver 75.

Torque from the instrument driver 75 is transmitted down the elongated shaft 71 using tendons along the elongated shaft 71. These individual tendons, such as pull wires, may be individually anchored to individual drive inputs 73 within the instrument handle 72. From the handle 72, the tendons are directed down one or more pull lumens along the elongated shaft 71 and anchored at the distal portion of the elongated shaft 71, or in the wrist at the distal portion of the elongated shaft. During a surgical procedure, such as a laparoscopic, endoscopic or hybrid procedure, these tendons may be coupled to a distally mounted end effector, such as a wrist, grasper, or scissor. Under such an arrangement, torque exerted on drive inputs 73 would transfer tension to the tendon, thereby causing the end effector to actuate in some way. In some embodiments, during a surgical procedure, the tendon may cause a joint to rotate about an axis, thereby causing the end effector to move in one direction or another. Alternatively, the tendon may be connected to one or more jaws of a grasper at the distal end of the elongated shaft 71, where tension from the tendon causes the grasper to close.

In endoscopy, the tendons may be coupled to a bending or articulating section positioned along the elongated shaft 71 (e.g., at the distal end) via adhesive, control ring, or other mechanical fixation. When fixedly attached to the distal end of a bending section, torque exerted on the drive inputs 73 would be transmitted down the tendons, causing the softer, bending section (sometimes referred to as the articulable section or region) to bend or articulate. Along the non-bending sections, it may be advantageous to spiral or helix the individual pull lumens that direct the individual tendons along (or inside) the walls of the endoscope shaft to balance the radial forces that result from tension in the pull wires. The angle of the spiraling and/or spacing therebetween may be altered or engineered for specific purposes, wherein tighter spiraling exhibits lesser shaft compression under load forces, while lower amounts of spiraling results in greater shaft compression under load forces, but limits bending. On the other end of the spectrum, the pull lumens may be directed parallel to the longitudinal axis of the elongated shaft 71 to allow for controlled articulation in the desired bending or articulable sections.

In endoscopy, the elongated shaft 71 houses a number of components to assist with the robotic procedure. The shaft 71 may comprise a working channel for deploying surgical tools (or medical instruments), irrigation, and/or aspiration to the operative region at the distal end of the shaft 71. The shaft 71 may also accommodate wires and/or optical fibers to transfer signals to/from an optical assembly at the distal tip, which may include an optical camera. The shaft 71 may also accommodate optical fibers to carry light from proximally-located light sources, such as light emitting diodes, to the distal end of the shaft 71.

At the distal end of the instrument 70, the distal tip may also comprise the opening of a working channel for delivering tools for diagnostic and/or therapy, irrigation, and aspiration to an operative site. The distal tip may also include a port for a camera, such as a fiberscope or a digital camera, to capture images of an internal anatomical space. Relatedly, the distal tip may also include ports for light sources for illuminating the anatomical space when using the camera.

In the example of FIG. 16, the drive shaft axes, and thus the drive input axes, are orthogonal to the axis of the elongated shaft 71. This arrangement, however, complicates roll capabilities for the elongated shaft 71. Rolling the elongated shaft 71 along its axis while keeping the drive inputs 73 static results in undesirable tangling of the tendons as they extend off the drive inputs 73 and enter pull lumens within the elongated shaft 71. The resulting entanglement of such tendons may disrupt any control algorithms intended to predict movement of the flexible elongated shaft 71 during an endoscopic procedure.

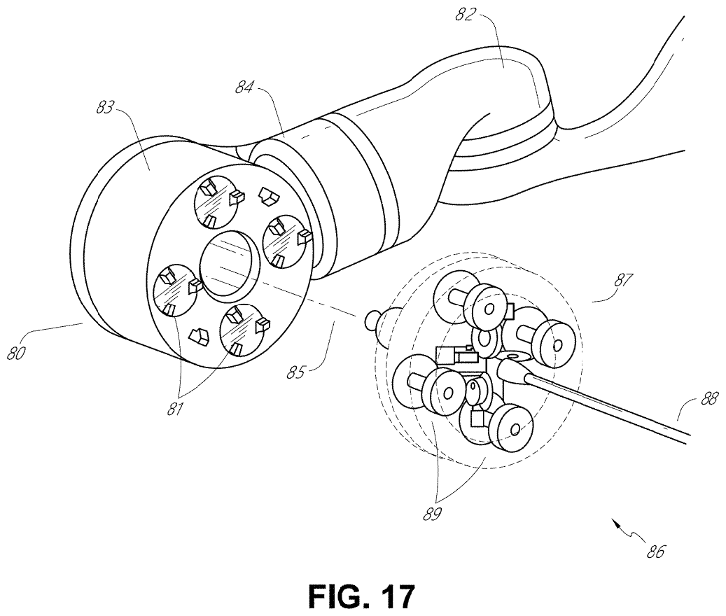

FIG. 17 illustrates an alternative design for an instrument driver and instrument where the axes of the drive units are parallel to the axis of the elongated shaft of the instrument. As shown, a circular instrument driver 80 comprises four drive units with their drive outputs 81 aligned in parallel at the end of a robotic arm 82. The drive units, and their respective drive outputs 81, are housed in a rotational assembly 83 of the instrument driver 80 that is driven by one of the drive units within the assembly 83. In response to torque provided by the rotational drive unit, the rotational assembly 83 rotates along a circular bearing that connects the rotational assembly 83 to the non-rotational portion 84 of the instrument driver 80. Power and controls signals may be communicated from the non-rotational portion 84 of the instrument driver 80 to the rotational assembly 83 through electrical contacts that may be maintained through rotation by a brushed slip ring connection (not shown). In other embodiments, the rotational assembly 83 may be responsive to a separate drive unit that is integrated into the non-rotatable portion 84, and thus not in parallel to the other drive units. The rotational mechanism 83 allows the instrument driver 80 to rotate the drive units, and their respective drive outputs 81, as a single unit around an instrument driver axis 85.

Like earlier disclosed embodiments, an instrument 86 may comprise an elongated shaft portion 88 and an instrument base 87 (shown with a transparent external skin for discussion purposes) comprising a plurality of drive inputs 89 (such as receptacles, pulleys, and spools) that are configured to receive the drive outputs 81 in the instrument driver 80. Unlike prior disclosed embodiments, the instrument shaft 88 extends from the center of the instrument base 87 with an axis substantially parallel to the axes of the drive inputs 89, rather than orthogonal as in the design of FIG. 16.

When coupled to the rotational assembly 83 of the instrument driver 80, the medical instrument 86, comprising instrument base 87 and instrument shaft 88, rotates in combination with the rotational assembly 83 about the instrument driver axis 85. Since the instrument shaft 88 is positioned at the center of instrument base 87, the instrument shaft 88 is coaxial with instrument driver axis 85 when attached. Thus, rotation of the rotational assembly 83 causes the instrument shaft 88 to rotate about its own longitudinal axis. Moreover, as the instrument base 87 rotates with the instrument shaft 88, any tendons connected to the drive inputs 89 in the instrument base 87 are not tangled during rotation. Accordingly, the parallelism of the axes of the drive outputs 81, drive inputs 89, and instrument shaft 88 allows for the shaft rotation without tangling any control tendons.

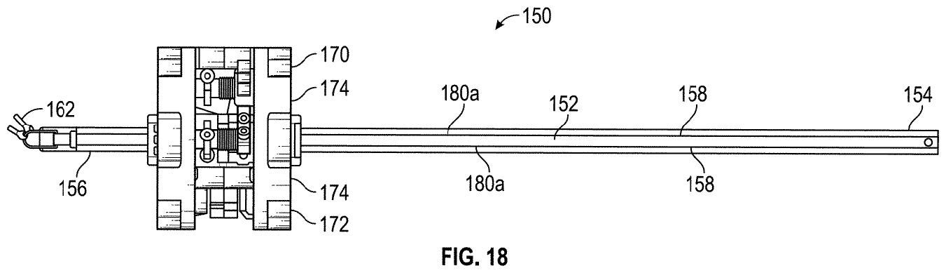

FIG. 18 illustrates an instrument having an instrument based insertion architecture in accordance with some embodiments. The instrument 150 can be coupled to any of the instrument drivers discussed above. The instrument 150 comprises an elongated shaft 152, an end effector 162 connected to the shaft 152, and a handle 170 coupled to the shaft 152. The elongated shaft 152 comprises a tubular member having a proximal portion 154 and a distal portion 156. The elongated shaft 152 comprises one or more channels or grooves 158 along its outer surface. The grooves 158 are configured to receive one or more wires or cables 180 therethrough. One or more cables 180 thus run along an outer surface of the elongated shaft 152. In other embodiments, cables 180 can also run through the elongated shaft 152. Manipulation of the one or more cables 180 (e.g., via an instrument driver) results in actuation of the end effector 162.

The instrument handle 170, which may also be referred to as an instrument base, may generally comprise an attachment interface 172 having one or more mechanical inputs 174, e.g., receptacles, pulleys or spools, that are designed to be reciprocally mated with one or more torque couplers on an attachment surface of an instrument driver. In some embodiments, the instrument 150 comprises a series of pulleys or cables that enable the elongated shaft 152 to translate relative to the handle 170. In other words, the instrument 150 itself comprises an instrument-based insertion architecture that accommodates insertion of the instrument, thereby minimizing the reliance on a robot arm to provide insertion of the instrument 150. In other embodiments, a robotic arm can be largely responsible for instrument insertion.

### E. Controller

Any of the robotic systems described herein can include an input device or controller for manipulating an instrument attached to a robotic arm. In some embodiments, the controller can be coupled (e.g., communicatively, electronically, electrically, wirelessly and/or mechanically) with an instrument such that manipulation of the controller causes a corresponding manipulation of the instrument e.g., via master slave control.

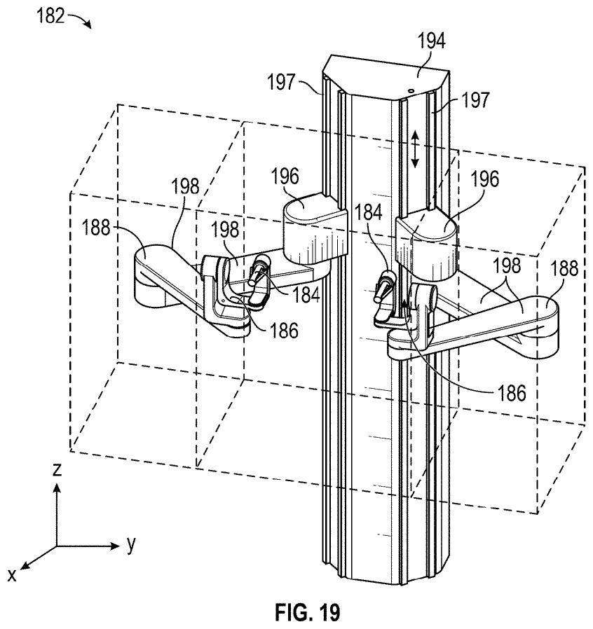

FIG. 19 is a perspective view of an embodiment of a controller 182. In the present embodiment, the controller 182 comprises a hybrid controller that can have both impedance and admittance control. In other embodiments, the controller 182 can utilize just impedance or passive control. In other embodiments, the controller 182 can utilize just admittance control. By being a hybrid controller, the controller 182 advantageously can have a lower perceived inertia while in use.

In the illustrated embodiment, the controller 182 is configured to allow manipulation of two medical instruments, and includes two handles 184. Each of the handles 184 is connected to a gimbal 186. Each gimbal 186 is connected to a positioning platform 188.

As shown in FIG. 19, each positioning platform 188 includes a SCARA arm (selective compliance assembly robot arm) 198 coupled to a column 194 by a prismatic joint 196. The prismatic joints 196 are configured to translate along the column 194 (e.g., along rails 197) to allow each of the handles 184 to be translated in the z-direction, providing a first degree of freedom. The SCARA arm 198 is configured to allow motion of the handle 184 in an x-y plane, providing two additional degrees of freedom.

In some embodiments, one or more load cells are positioned in the controller. For example, in some embodiments, a load cell (not shown) is positioned in the body of each of the gimbals 186. By providing a load cell, portions of the controller 182 are capable of operating under admittance control, thereby advantageously reducing the perceived inertia of the controller while in use. In some embodiments, the positioning platform 188 is configured for admittance control, while the gimbal 186 is configured for impedance control. In other embodiments, the gimbal 186 is configured for admittance control, while the positioning platform 188 is configured for impedance control. Accordingly, for some embodiments, the translational or positional degrees of freedom of the positioning platform 188 can rely on admittance control, while the rotational degrees of freedom of the gimbal 186 rely on impedance control.

### F. Navigation and Control

Traditional endoscopy may involve the use of fluoroscopy (e.g., as may be delivered through a C-arm) and other forms of radiation-based imaging modalities to provide endoluminal guidance to an operator physician. In contrast, the robotic systems contemplated by this disclosure can provide for non-radiation-based navigational and localization means to reduce physician exposure to radiation and reduce the amount of equipment within the operating room. As used herein, the term “localization” may refer to determining and/or monitoring the position of objects in a reference coordinate system. Technologies such as preoperative mapping, computer vision, real-time EM tracking, and robot command data may be used individually or in combination to achieve a radiation-free operating environment. In other cases, where radiation-based imaging modalities are still used, the preoperative mapping, computer vision, real-time EM tracking, and robot command data may be used individually or in combination to improve upon the information obtained solely through radiation-based imaging modalities.

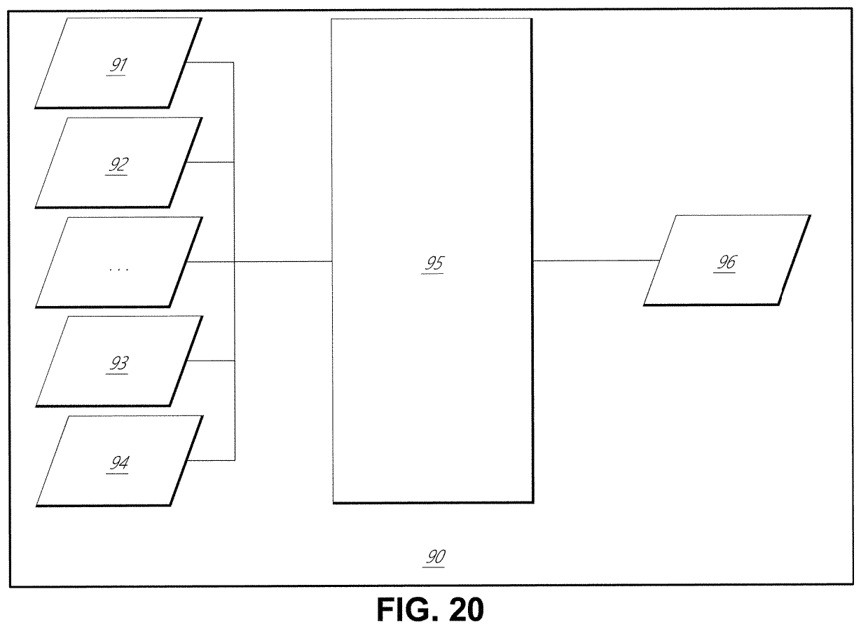

FIG. 20 is a block diagram illustrating a localization system 90 that estimates a location of one or more elements of the robotic system, such as the location of the instrument, in accordance to an example embodiment. The localization system 90 may be a set of one or more computer devices configured to execute one or more instructions. The computer devices may be embodied by a processor (or processors) and computer-readable memory in one or more components discussed above. By way of example and not limitation, the computer devices may be in the tower 30 shown in FIG. 1, the cart 11 shown in FIGS. 1-4, the beds shown in FIGS. 5-14, etc.

As shown in FIG. 20, the localization system 90 may include a localization module 95 that processes input data 91-94 to generate location data 96 for the distal tip of a medical instrument. The location data 96 may be data or logic that represents a location and/or orientation of the distal end of the instrument relative to a frame of reference. The frame of reference can be a frame of reference relative to the anatomy of the patient or to a known object, such as an EM field generator (see discussion below for the EM field generator).

The various input data 91-94 are now described in greater detail. Preoperative mapping may be accomplished through the use of the collection of low dose CT scans. Preoperative CT scans are reconstructed into three-dimensional images, which are visualized, e.g. as “slices” of a cutaway view of the patient's internal anatomy. When analyzed in the aggregate, image-based models for anatomical cavities, spaces and structures of the patient's anatomy, such as a patient lung network, may be generated. Techniques such as center-line geometry may be determined and approximated from the CT images to develop a three-dimensional volume of the patient's anatomy, referred to as model data 91 (also referred to as “preoperative model data” when generated using only preoperative CT scans). The use of center-line geometry is discussed in U.S. Pat. App. Ser. No. 14/523,760, the contents of which are herein incorporated in its entirety. Network topological models may also be derived from the CT-images, and are particularly appropriate for bronchoscopy.

In some embodiments, the instrument may be equipped with a camera to provide vision data (or image data) 92. The localization module 95 may process the vision data 92 to enable one or more vision-based (or image-based) location tracking modules or features. For example, the preoperative model data 91 may be used in conjunction with the vision data 92 to enable computer vision-based tracking of the medical instrument (e.g., an endoscope or an instrument advance through a working channel of the endoscope). For example, using the preoperative model data 91, the robotic system may generate a library of expected endoscopic images from the model based on the expected path of travel of the endoscope, each image linked to a location within the model. Intraoperatively, this library may be referenced by the robotic system in order to compare real-time images captured at the camera (e.g., a camera at a distal end of the endoscope) to those in the image library to assist localization.

Other computer vision-based tracking techniques use feature tracking to determine motion of the camera, and thus the endoscope. Some features of the localization module 95 may identify circular geometries in the preoperative model data 91 that correspond to anatomical lumens and track the change of those geometries to determine which anatomical lumen was selected, as well as the relative rotational and/or translational motion of the camera. Use of a topological map may further enhance vision-based algorithms or techniques.

Optical flow, another computer vision-based technique, may analyze the displacement and translation of image pixels in a video sequence in the vision data 92 to infer camera movement. Examples of optical flow techniques may include motion detection, object segmentation calculations, luminance, motion compensated encoding, stereo disparity measurement, etc. Through the comparison of multiple frames over multiple iterations, movement and location of the camera (and thus the endoscope) may be determined.

The localization module 95 may use real-time EM tracking to generate a real-time location of the endoscope in a global coordinate system that may be registered to the patient's anatomy, represented by the preoperative model. In EM tracking, an EM sensor (or tracker) comprising one or more sensor coils embedded in one or more locations and orientations in a medical instrument (e.g., an endoscopic tool) measures the variation in the EM field created by one or more static EM field generators positioned at a known location. The location information detected by the EM sensors is stored as EM data 93. The EM field generator (or transmitter), may be placed close to the patient to create a low intensity magnetic field that the embedded sensor may detect. The magnetic field induces small currents in the sensor coils of the EM sensor, which may be analyzed to determine the distance and angle between the EM sensor and the EM field generator. These distances and orientations may be intraoperatively “registered” to the patient anatomy (e.g., the preoperative model) in order to determine the geometric transformation that aligns a single location in the coordinate system with a position in the preoperative model of the patient's anatomy. Once registered, an embedded EM tracker in one or more positions of the medical instrument (e.g., the distal tip of an endoscope) may provide real-time indications of the progression of the medical instrument through the patient's anatomy.

Robotic command and kinematics data 94 may also be used by the localization module 95 to provide localization data 96 for the robotic system. Device pitch and yaw resulting from articulation commands may be determined during preoperative calibration. Intraoperatively, these calibration measurements may be used in combination with known insertion depth information to estimate the position of the instrument. Alternatively, these calculations may be analyzed in combination with EM, vision, and/or topological modeling to estimate the position of the medical instrument within the network.

As FIG. 20 shows, a number of other input data can be used by the localization module 95. For example, although not shown in FIG. 20, an instrument utilizing shape-sensing fiber can provide shape data that the localization module 95 can use to determine the location and shape of the instrument.

The localization module 95 may use the input data 91-94 in combination(s). In some cases, such a combination may use a probabilistic approach where the localization module 95 assigns a confidence weight to the location determined from each of the input data 91-94. Thus, where the EM data may not be reliable (as may be the case where there is EM interference) the confidence of the location determined by the EM data 93 can be decrease and the localization module 95 may rely more heavily on the vision data 92 and/or the robotic command and kinematics data 94.

As discussed above, the robotic systems discussed herein may be designed to incorporate a combination of one or more of the technologies above. The robotic system's computer-based control system, based in the tower, bed and/or cart, may store computer program instructions, for example, within a non-transitory computer-readable storage medium such as a persistent magnetic storage drive, solid state drive, or the like, that, upon execution, cause the system to receive and analyze sensor data and user commands, generate control signals throughout the system, and display the navigational and localization data, such as the position of the instrument within the global coordinate system, anatomical map, etc.

### 2. Robotically-Actuated Medical Retractors

The robotic medical systems described above with reference to FIGS. 1-20, as well as other robotic medical systems, can be used to perform a variety of medical procedures, including both laparoscopic, endoscopic, and open procedures. During such procedures, medical retractors can be used to support or hold organs, tissue, or other objects that may obscure or are located in proximity to a target treatment region so as to allow better access to the target treatment region. As will be described in this section, robotically-actuated medical retractors can be used in combination with the robotic medical systems described above (and others) to provide several advantages over prior systems and manual medical retractors. In this section, liver retractors will be described as the primary example, with the understanding that other types of medical retractors can also be used in other situations.

Liver retractors are commonly used to support a patient's liver to allow access to other target anatomy within the patient's abdominal cavity. Many upper abdominal procedures require liver retraction in order to obtain adequate exposure of the target anatomy. Minimally invasive procedures that utilize liver retraction include, for example, cholecystectomies (with approximately 414,000 U.S. procedures per year (“PPY”)), Nissen fundoplications (with approximately 35,000 U.S. PPY), gastric bypasses (with approximately 94,800 U.S. PPY), and sleeve gastrectomy (with approximately 92,400 U.S. PPY).

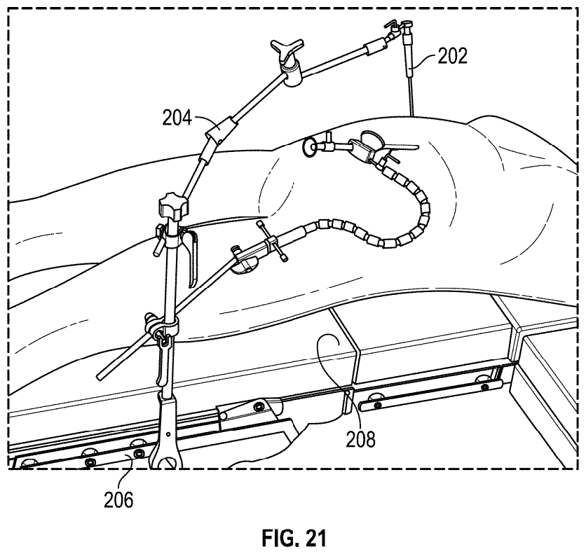

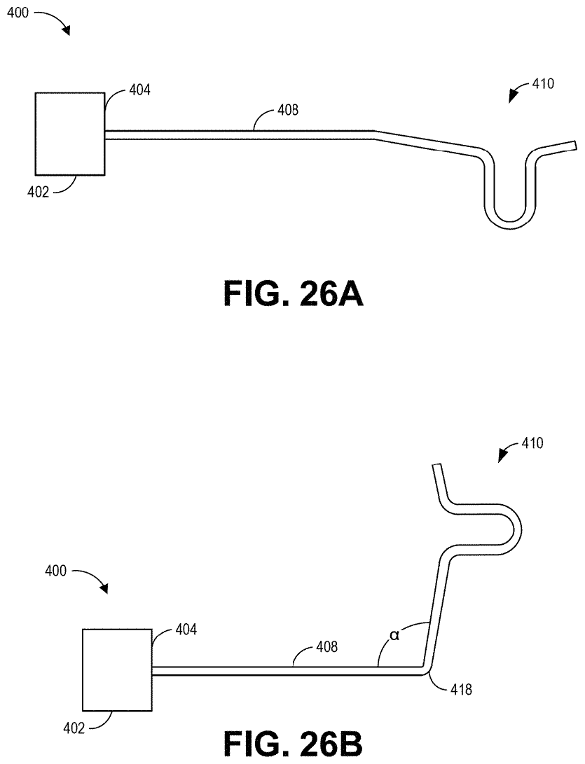

Generally during such procedures, liver retractors or similar instruments are manually manipulated into position and then attached to a surgical table rail as shown in FIG. 21, which illustrates an example of a manual liver retractor 202 during use. As shown in FIG. 21, the manual liver retractor 202 has been inserted into the patient (e.g., via a laparoscopic port) and manipulated such that it can hold, move, or support the patient's liver within the patient's abdominal cavity. In FIG. 21, a distal portion of the liver retractor 202 (e.g., the portion supporting the liver) is not visible as it is positioned within the patient's abdominal cavity. Generally, the distal portion of liver retractor 202 comprises a shape that can be inserted through a small incision in the patient's abdomen and that also provides a large surface area or other suitable shape for supporting the liver. One example of such a shape is an open loop shape as will be described in greater detail with reference to FIGS. 26A and 26B.

As shown in FIG. 21, for example, once the liver retractor 202 has been positioned to support the liver, it is often attached to a table mount 204 that is configured to hold the liver retractor 202 in place during the remainder of the procedure. The table mount 204 can be attached to a rail 206 of a surgical table or platform 208 that supports the patient as shown in FIG. 21, or the table mount 204 can attach to another object or support stand in the operating environment. In the illustrated embodiment, the table mount 204 includes a plurality of articulable joints such that the shape or pose of the table mount 204 can be adjusted to facilitate positioning of the liver retractor 202. In the case of a manual liver retractor 202, the articulable joints of the table mount 204 are manually adjustable, and the physician or assistant manually manipulates the joints of the table mount 204 to get it into the desired position. Once the table mount 204 is manually manipulated into position and the liver retractor 202 is attached thereto, both the table mount 204 and the liver retractor 202 are set in place. If further adjustments are desired, the physician or assistant again manually manipulates them to re-position them into a new configuration. In cases where the liver or liver retractor 202 needs to be repositioned during surgery, this manual repositioning can be time consuming and tedious.

In the context of robotic medical procedures, such as those that can be performed using the robotic medical systems described above with reference to FIGS. 1-20, robotic control of a liver retractor can be desirable. For example, during a robotic medical procedures, the operating physician may control a robotic medical system remotely, for example, remotely controlling one or more surgical tools that can be attached to one or more robotic arms. Robotic control of a liver retractor can allow adjustments to the retraction (e.g., to the position of the liver retractor 202) without requiring the operating physician to pause his or her operation, sterilize (scrub in) to enter the surgical site from a remote location, and/or verbally communicate adjustment instructions to an assistant or sterile staff personnel who may not be sufficiently trained to make the adjustments safely. Rather, use of a robotically-controllable liver retractor may advantageously allow the operator to remotely control the liver retractor in a manner similar to the other robotically-controllable surgical tools.

Additionally or alternatively, robotic control of a liver retractor can enhance patient safety or awareness of a robotic system. For example, the robotic system may be capable of determining a position and orientation of each robotically controlled component based on acquired kinematic data (e.g., using an encoder and/or sensor in each joint of each robotic arm) and prior knowledge of the structural dimensions of the system (e.g., a length of each linkage in each robotic arm). Such positional awareness can be useful in providing sufficient freedom of operation while avoiding inadvertent collisions between robotic arms and robotically controlled instruments. However, the robotic arms and instruments of the robotic medical system may be prone to collide with elements outside of the control and awareness of the robotic system, such as the liver retractor 202 or the table mount 204 in embodiments where the liver retractor and/or table mount are purely manually controlled. This is especially problematic for robotic arms that are unable to detect the force of a collision with the liver retractor 202 or table mount 204. This may be dangerous for the patient, as the collision could inadvertently move the liver retractor 202 and cause a major vessel perforation, parenchymal damage, ischemia-caused necrosis, diaphragm perforation and injury to thoracic tissue, or other harmful outcomes. Further, since the physician at the surgeon console might not be provided feedback on external forces on the robotic arms, visual cues might be the only way the user would know they are in a potentially hazardous situation.

Embodiments disclosed herein provide for robotically-controlled and actuated liver retractors. While presented in the context of a liver retractor, the medical retractors described herein can be configured for use with other organs, tissues, or objects. As will be described more fully below, the robotically-actuated liver retractors can allow a physician to actively manage his or her retraction dynamically throughout a procedure, in order to ensure optimal exposure of the target site at all times. That is, in some embodiments, the physician is able to remotely control the position of the liver retractor in a manner that is similar to how the physician can control the other robotically-enabled medical instruments during the procedure. This robotically-controlled solution avoids, prevents, or reduces the likelihood of the issues associated with requiring the physician or a sterile assistant to manually adjust the retractor and those associated with accidental contact between the robotic arms and the retractor.

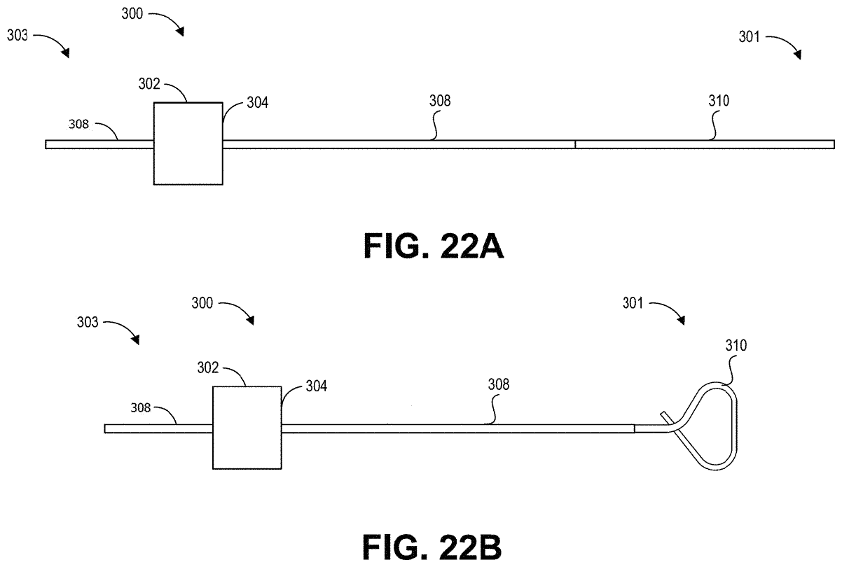

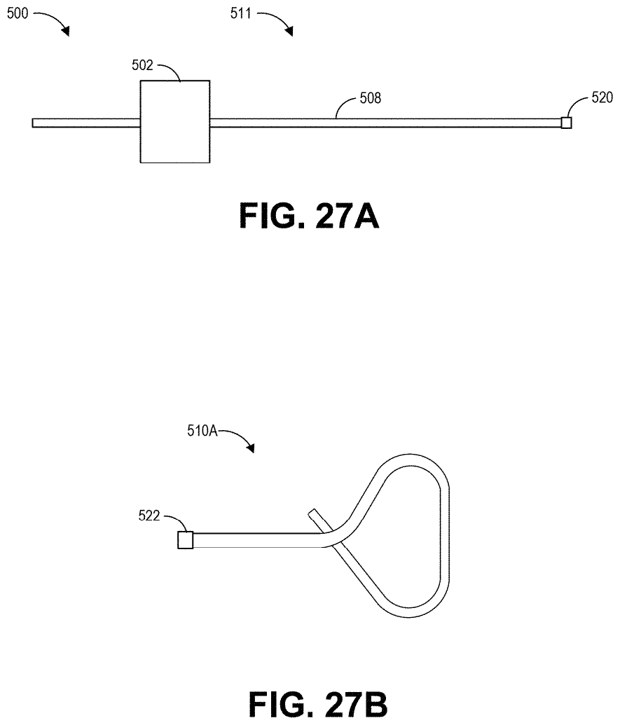

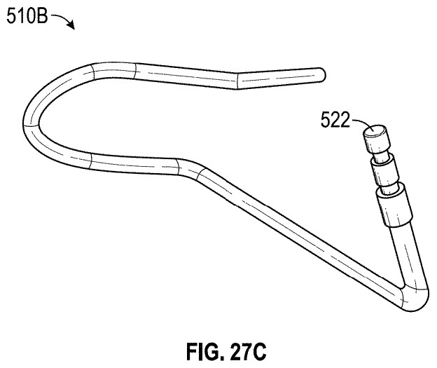

In some embodiments, the robotically-actuated liver retractors can be a fixed or non-articulable, zero degree of freedom instrument (as shown in FIGS. 26A and 26B, described below) or an articulable single- or multi- degree of freedom articulating instrument (as shown in FIGS. 22A-25B). Further, in some embodiments, the robotically-actuated liver retractors can be configured such that a distal portion thereof (e.g., a portion of the liver retractor that is configured to be inserted into the patient) is removable, replaceable, and/or disposable (as shown in FIGS. 27A-27C, described below). To further illustrate the functionality and features of the robotically-actuated liver retractors, several examples will now be described with reference to the figures. The embodiments illustrated in the figures are provided by way of example and should not be construed as limiting.

FIGS. 22A and 22B illustrate an embodiment of a robotically-actuated medical retractor 300. In the illustrated embodiment, the retractor 300 is configured as a liver retractor. That is, in the illustrated embodiment, the retractor 300 is configured to support a liver during a medical procedure. The retractor 300 illustrated in FIGS. 22A and 22B is an articulating retractor because, as will be described in further detail below, a distal portion 310 of the retractor 300 is configured to articulate (e.g., change shape, change pose, bend, fold, etc.). FIG. 22A illustrates the retractor 300 with the distal portion 310 in an unarticulated configuration, and FIG. 22B illustrates the retractor 300 with the distal portion 310 in an articulated configuration. In some embodiments, the unarticulated configuration (FIG. 22A) is configured to facilitate insertion of the distal portion 310 of the retractor 300 into the patient, and the articulated configuration (FIG. 22B) is configured to support the patient's liver. Further, although FIG. 22B illustrates one articulated configuration, other articulated configurations may also be possible.

As shown in FIGS. 22A and 22B, the retractor 300 comprises an instrument base 302 (which can also be referred to as an instrument handle). The base 302 can be configured to selectively attach or couple to an actuating and/or positioning device, such as a robotic arm. Thus, the base 302 can be configured to allow the retractor 300 to selectively attach or couple with a robotic arm. Once the base 302 is attached to the robotic arm, the robotic arm can be manipulated (e.g., actuated, moved, or articulated) to control the position of the retractor 300. This can allow the retractor 300 to be robotically repositioned during the medical procedure in a manner that is not possible with manual liver retractors. In some embodiments, attachment of the base 302 to the robotic arm need not be direct. One or more additional components can be positioned between the base 302 and the robotic arm. For example, in some embodiments, the base 302 attaches to a sterile adapter that is positioned on or attached to the robotic arm. In some embodiments, the sterile adapter is first attached to the robotic arm and then the base 302 is attached to the sterile adapter. The reverse may also be true. In some embodiments, a sterile adapter can be attached to the base 302 and then the base 302 and sterile adapter can be attached to the robotic arm. The sterile adapter can provide a sterile barrier between the robotic arm and the base 302.

The base 302 can include an attachment surface or face 304 configured to couple or attach to the robotic arm. The attachment face 304 can be located on any side of the base 302. In the illustrated embodiment, the attachment face 304 is located on a distal side of the base 302 (e.g., the side of the base 302 facing the distal end 301 of the retractor 300 or the side of the base 302 facing towards the patient during a procedure). This configuration may advantageously allow the retractor 300 to be top loaded or through loaded onto the end of the robotic arm. This can allow the retractor 300 to be removed by pulling the base 302 away from the robotic arm in a proximal direction (e.g., away from the patient's body) which can improve patient safety during the procedure. Although, illustrated on the distal side of the base 302, the attachment face 304 can be located on any side of the base 302 in other embodiments, such as on a proximal side, for example.

As will be described in further detail below with reference to FIG. 23, the base 302 may also include one or more instrument drive inputs. The instrument drive inputs can be actuated (e.g., rotated or otherwise operated) to actuate or control various functionality of the retractor 300. For example, the instrument drive inputs can be actuated to cause articulation of the distal portion 310 of the retractor 300. In the illustrated embodiment, the instrument drive inputs can be used to articulate the distal portion 310 of the retractor between the unarticulated configuration (FIG. 22A) and the articulated configuration (FIG. 22B) and vice versa.

The one or more instrument drive inputs can be positioned on the attachment face 304. This can allow the one or more instrument drive inputs to engage with corresponding one or more instrument drive outputs of the robotic arm (or other robotic device to which the retractor 300 is attached). In some embodiments, the robotic arm includes an instrument driver or instrument drive mechanism as described above with reference to FIGS. 15-16. The instrument driver can include the one or more instrument drive outputs that are configured to engage with and actuate the one or more instrument drive inputs when the base 302, when the base 302 is coupled to the instrument driver of the robotic arm. In this way, the position of the retractor 300 can be adjusted by moving the base 302 with the robotic arm, and the articulation of the distal portion 310 of the retractor 300 can be controlled using the robotic drive outputs on the instrument driver.

As shown in FIGS. 22A and 22B, the retractor 300 also includes a shaft 308 extending from the base 302. In some embodiments, the shaft 308 can be an elongated shaft and/or a rigid shaft. In the illustrated embodiment, the shaft 308 extends through base 302. This configuration can be beneficial where the base 302 is configured to drive insertion and/or retraction of the shaft 308 relative to the base 302. An example of such an insertion/retraction architecture is described in more detail above with reference to the embodiment illustrated in FIG. 18. Further, such insertion and/or retraction of the shaft 308 relative to the base 302 can be drive by engagement of the one or more instrument drive inputs of the base 302 with the one or more instrument drive outputs of the instrument driver. Configuring the retractor 300 such that the shaft 308 can insert and/or retract relative to the base 302 can advantageously allow the retractor 300 to be repositioned (e.g., inserted or withdrawn) without requiring movement of the robotic arm. For example, in some embodiments, a depth of the distal portion 310 of the retractor 300 within the patient can be adjusted by inserting or retracting the shaft 308 relative to the base 302 without requiring movement of the robotic arm.

In the illustrated embodiment, the distal portion 310 of the retractor 300 comprises a distal portion of the shaft 308. In the illustrated embodiment, the distal portion 310 is configured to articulate as mentioned above. For example, the distal portion 310 of the shaft 308 can be configured to articulate between a substantially linear configuration (for example, as shown in FIG. 22A) and a bent configuration for atraumatically supporting a liver during a robotic medical procedure (for example, as shown in FIG. 22B). The substantially linear configuration can facilitate insertion of the distal portion 310 into the patient. For example, when in the substantially linear configuration, the distal portion 310 can be inserted through a small surgical incision or port. Once the distal portion 310 is positioned within the patient, it can be articulated into the bent configuration, which can be configured to atraumatically support a liver.

The bent configuration of the distal portion 310 can take many shapes. For example, in the illustrated embodiment of FIG. 22B, the bent configuration of the distal portion 310 comprises a closed loop shape. In the illustrated embodiment, the closed loop shape is a generally triangular shape, but other closed loop shapes are also possible, including, square, rectangular, oval, circular, etc. As used herein, a “closed loop” shape refers to a bent or curved shape in which an end or other portion of the bent shape crosses over or connects with a start or other portion of the bent shape. For example, in FIG. 22B, the distal portion 310 is configured such that a distal end of the distal portion 310 crosses over a proximal end of the distal portion 310 so as to form a closed loop triangular shape. In some embodiments, the bent configuration of the distal portion 310 can comprise an open loop shape, such as a shape similar to the open loop configuration shown in FIGS. 26A and 26B, for example. In general, the bent configuration is configured as a shape that is configured to atraumatically support the liver or another organ, tissue, or object during a medical procedure. In some embodiments, to facilitate atraumatic support, the distal portion 310 of the retractor 300 can configured as a bar having rounded outer surfaces (e.g., a generally circular cross-section or other cross-sectional shape forming an outer diameter with rounded edges) and a rounded tip.

In some embodiments, to enable articulation of the distal portion 310 of the shaft 308 of the retractor 300, the retractor 300 can include at least one tendon or pull wire extending between an instrument drive input of the base 302 and the distal portion 310 of the shaft 308. The pull wire can be configured to articulate the distal portion 310 as described in further detail below with reference to the examples of FIGS. 23-25B.

FIG. 23 illustrates an embodiment of the base 302 of the robotically-actuated retractor 300. In this perspective view, the attachment face 304 of the base 302 can be seen. As shown, in this example, the attachment face 304 includes five instrument drive inputs 312. Other numbers of drive inputs (e.g., one, two, three, four, or more) are possible in other embodiments. The instrument drive inputs 312 can be configured to engage corresponding drive outputs on an instrument driver to control various functionality of the retractor 300 as mentioned previously.

In the illustrated embodiment, the instrument drive inputs 312 comprise receptacles configured to engage (e.g., receive and/or mesh with) instrument drive outputs that are configured as splines. In the illustrated embodiment, the instrument drive inputs 312 are configured to rotate when driven by corresponding rotation of the instrument drive outputs. The reverse may also be true. The instrument drive inputs 312 can be configured as splines and the instrument drive outputs can be configured as receptacles. In other embodiments, the instrument drive inputs 312 and the instrument drive outputs can comprise other structures configured to mechanically transfer motion therebetween.

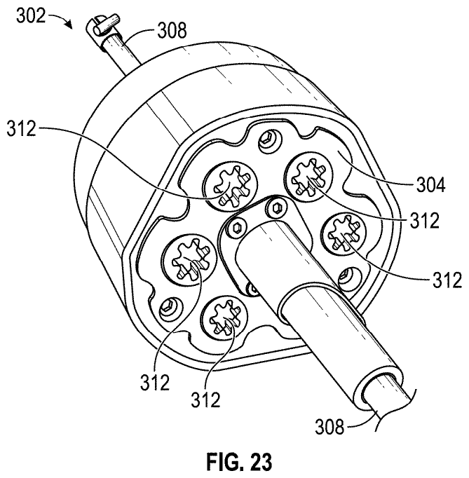

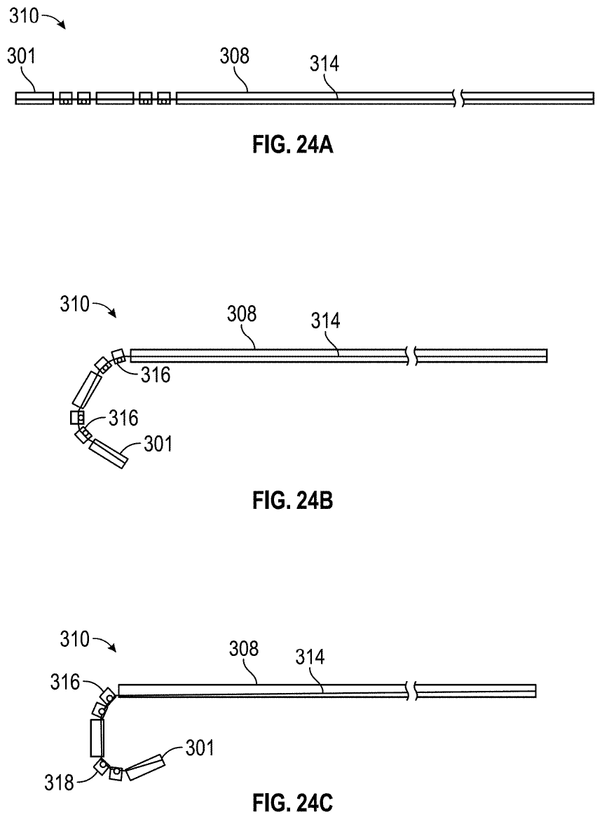

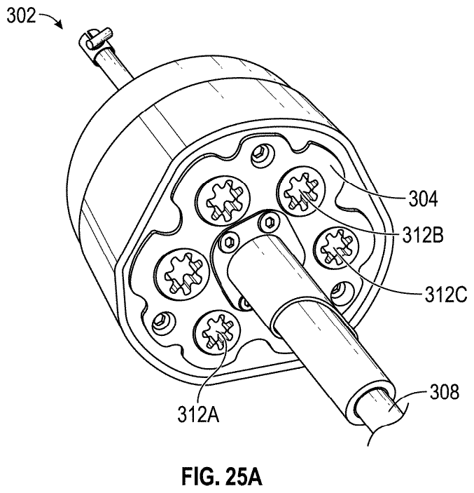

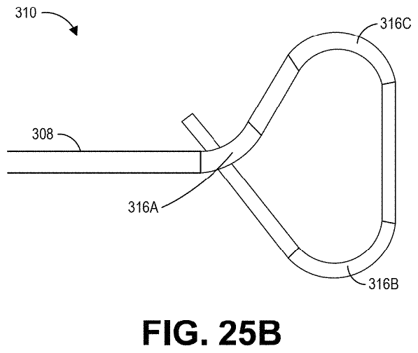

As mentioned previously, the instrument drive inputs 312 can be configured to allow control or actuation of various functionality of the retractor 300. For example, one or more of the instrument drive inputs 312 can be configured to control articulation of the distal portion 310 of the elongated shaft 308. In some embodiments, a single instrument drive input 312 can be configured to control articulation of the distal portion 310. An example of this will be described in further detail below with reference to FIGS. 24A-24C. In other embodiments, multiple instrument drive inputs 312 can be configured to control articulation of the distal portion 310. For example, the distal portion 310 can comprise a plurality of independently articulable sections, with actuation of each section corresponding to one of a plurality of drive inputs 312. In some embodiments, each one of the drive inputs 312 can be operable to independently articulate a respective one of the articulable sections. An example of this will be described in further detail below with reference to FIGS. 25A and 25B.

In some embodiments, one or more of the instrument drive inputs 312 can be configured to control insertion and/or retraction of the shaft 308 relative to the base 302. For example, in some embodiments, rotating one of the instrument drive inputs 312 in a first direction can drive insertion of the shaft 308 and rotating the instrument drive input 312 in a second, opposite direction can drive retraction of the shaft 308. In some embodiments, one of the instrument drive inputs 312 can be rotated to drive insertion of the shaft 308 relative to the base 302 and another one of the instrument drive inputs 312 can be rotated to drive retraction of the shaft 308 relative to the base 302 (e.g., according to the instrument based insertion architecture described above with reference to FIG. 18). In such embodiments, insertion or retraction of the shaft 308 can involve axial motion of the shaft relative to the instrument base, in which the shaft translates relative to the instrument base along its longitudinal axis.

In some embodiments, the shaft 308 is configured to roll (e.g., rotate about its longitudinal axis) relative to the base 302. In such embodiments, one or more of the instrument drive inputs 312 can be configure to control roll of the shaft 308 relative to the base 302, such that operation of the drive input(s) 312 causes the shaft 308 to rotate about its longitudinal axis. For example, in some embodiments, rotating one of the instrument drive inputs 312 in a first direction can drive roll of the shaft 308 in a first direction and rotating the instrument drive input 312 in a second, opposite direction can drive roll of the shaft 308 in a second, opposite direction. In some embodiments, one of the instrument drive inputs 312 can be rotated to drive roll of the shaft 308 relative to the base 302 in a first direction and another one of the instrument drive inputs 312 can be rotated to drive roll of the shaft 308 relative to the base 302 in a second direction.

The attachment face 304 can also include features configured to provide information about the retractor 300 to the robotic medical system to which it is attached. For example, a memory or other suitable structure or device can be included on or below the attachment face 304 that can be read by a corresponding reader on the robotic arm or instrument driver when the base 302 is attached. The memory or other device can store information about the retractor 300, such as information about the type of retractor, the model number, calibration information, etc. In an example, the attachment interface includes a radio-frequency identification (RFID) tag, which when the base 302 is attached to the robotic system, can be read by a corresponding reader.

FIGS. 24A-24C illustrate an embodiment of an articulable distal portion 310 of the robotically-actuated retractor 300. FIG. 24A illustrates the distal portion 310 in an unarticulated configuration, FIG. 24B illustrates the distal portion 310 in a first articulated configuration, and FIG. 24C illustrates the distal portion 310 in an second articulated configuration. In the illustrated embodiment, the retractor 300 includes a pull wire 314 configured to cause articulation of the distal portion 310. As shown in the illustrated example, the pull wire 314 extends down or along the shaft 308 to the distal end 301 (e.g., to the left relative to the orientation illustrated in the figure). The pull wire 314 can be attached at a proximal end to one of the instrument drive inputs 312. For example, the pull wire 314 can be wound on a pulley coupled to one of the instrument drive inputs 312 such that rotation of the instrument drive input 312 causes the pull wire 314 to spool or unspool from the pulley.

In the illustrated embodiment, the distal portion 310 of the shaft 308 includes two articulating sections 316 (FIGS. 24B and 24C). The two articulating section 316 can each be configured as sections that can bend to allow articulation of the distal portion 310. For example, in some embodiments, the articulating sections 316 include one or more joints. As another example, in some embodiments, the articulating sections 316 can be made from a flexible or bendable material. Although FIGS. 24A-24C illustrate an example with two articulating sections 316, other numbers of articulating sections 316 can be included in other embodiments. For example, the distal portion 310 can be configured to include one, two, three, four or more articulating sections 316.

FIGS. 24A-24C illustrate various stages in an example articulation process. FIG. 24A illustrates the distal portion 310 of the shaft 308 in an unarticulated or generally linear configuration. To cause articulation, tension can be placed on the pull wire 314. For example, the instrument drive input 312 associated with the pull wire 314 can be actuated to wind the pull wire 314 onto the pulley causing a change in tension or shortening in the pull wire 314. As the tension increases (or the pull wire 314 is shortened), the pull wire 314 causes the articulating sections 316 to bend. FIG. 24B illustrates a first stage in the articulation. As the tension in the pull wire 314 increases, the distal portion 310 of the shaft 308 continues to articulate. FIG. 24C illustrates a second stage in the articulation.

In this example, a single pull wire 314 is configured to drive articulation of the distal portion 310. That is, the single pull wire 314 is configured to cause articulation or bending of both articulating sections 316. This need not be the case in all embodiments. For example, as shown in FIGS. 25A and 25B, multiple pull wires 314 can be included that are configured to independently articulate the different articulating sections 316 of the distal portion 310 of the shaft 308 of the retractor.

FIGS. 25A and 25B illustrate an embodiment of a robotically-actuated medical retractor 300 that includes an articulable distal portion 310 (FIG. 25B) with three independently articulating sections 316A, 316B, 316C. FIG. 25A illustrates an embodiment of the base 302 of the medical retractor 300, and FIG. 25B illustrates an embodiment of the articulable distal portion 310 of the medical retractor in an articulated configuration. As shown in FIG. 25B, the distal portion 310 of the retractor 300 can include a first articulating section 316A, a second articulating section 316B, and a third articulating section 316C. In the illustrated embodiment, each articulating section 316 separates two adjacent rigid sections of the shaft 308. In some embodiments, each articulating section 316 is independently articulable. That is, articulating section 316A can be articulated independently of articulation sections 316B, 316C, for example. Configuring the distal portion 310 with independently articulable sections 316 can provide even greater control for the physician, allowing the physician to manipulate the distal portion 310 into a large number of different positions.

In some embodiments, to allow for independent actuation of the articulating sections 316A, 316B, 316C, the base 302 can include corresponding instrument drive inputs 312A, 312B, 312C. Each instrument drive input 312A, 312B, 312C can also be independently actuable. A first pull wire can extend between the drive input 312A and the articulating section 316A such that the drive input 312A can cause articulating of the articulating section 316A. A second pull wire can extend between the drive input 312B and the articulating section 316B such that the drive input 312B can cause articulating of the articulating section 316B. And a third pull wire can extend between the drive input 312C and the articulating section 316C such that the drive input 312C can cause articulating of the articulating section 316C. In some embodiments, one of the articulating sections 316 can be configured to bend in an different direction from another of the articulating sections 316. For example as seen in FIG. 25B, the first articulating section 316A can be configured to bend in a first direction, while the second articulating section 316B and third articulating section 316C are each configured to bend in a second direction opposite the first direction.

Although FIGS. 25A and 25B illustrate an embodiment with three articulating sections 316A, 316B, 316C independently actuated by three instrument drive inputs 312A, 312B, 312C, other numbers of articulating sections 316 and drive inputs 312 are possible. For example, the retractor 300 can include one, two, three, four, five or more articulating sections 316 and corresponding drive inputs 312.

FIGS. 26A and 26B illustrate embodiments of a robotically-actuated medical retractor 400 that includes a non-articulating or rigid distal portion 410 (in contrast with the retractor 300 previously described which included an articulable distal portion 310). With reference first to the embodiment shown in FIG. 26A, the retractor 400 comprises a base 402 and an elongated shaft 408. A distal portion 410 of the elongated shaft 408 is configured with a fixed or rigid shape that allows the distal portion 410 to be inserted into the patient and support an organ or tissue (such as the liver, for example).

Similar to the base 302, the base 402 can be configured to selectively attach or couple to a positioning device, such as a robotic arm. Thus, the instrument base 402 can be configured to allow the retractor 400 to selectively attach or couple with a robotic arm. Once the base 402 is attached to the robotic arm, the robotic arm can be manipulated (e.g., moved or articulated) to control the position of the retractor 400. This can allow the retractor 400 to be robotically repositioned during the medical procedure in a manner that is not possible with manual liver retractors. The base 402 can include an attachment surface or face 404 configured to couple or attach to the robotic arm as previously described.

The base 402 may also include one or more instrument drive inputs. The instrument drive inputs can be actuated (e.g., rotated or otherwise actuated) to actuate or control various functionality of the retractor 400. For example, the instrument drive inputs can be rotated to cause insertion or retraction of the shaft 408 and/or to drive roll of the shaft 408. The one or more instrument drive inputs can be positioned on the attachment face 404 as described above. As shown in FIG. 26A, the shaft 408 extends from the base 402. In some embodiments, the shaft 408 can be an elongated shaft and/or a rigid shaft. In the illustrated embodiment, the proximal end of the shaft 408 is connected to the base 402. However, in other embodiments, the shaft 408 can extend through the base 402 such that insertion or retraction of the shaft 408 can be driven relative to the base 402 as previously described.

The distal portion 410 of the shaft 408 can be rigid or fixed and configured with a shape for atraumatically supporting the patient's liver. For example, in the illustrated embodiment, distal portion 410 comprises an open loop shape. As illustrated, the open loop shape comprises a U-shape, but other open loop shapes are possible. As used herein, an “open loop” refers to a curved or bent structure in which gap remains between a start of the open loop and an end of the open loop. Examples of open loop shapes includes C-shaped bends, U-shaped bends, and hooks. In such shapes, the terminal end of the bent structure can be spaced apart from a start of the bend structure, such that a collective angle of the bent portion is less than 360 degrees. According to some embodiments, an open loop may be formed with a bend that is between 90 and 270 degrees so that the open loop collectively forms a sufficiently broad structure in which opposing ends of the open loop can atraumatically support a weight of a liver, but a sufficiently large gap is formed so that the open loop can be inserted through a small opening or incision on a patient. When the distal portion 410 is rigid, the open loop shape is beneficial because it allows the distal portion 410 to be inserted into the patient. For example, the distal tip of the distal portion 410 can first be inserted through a surgical incision or port and then the remainder of the shape can be worked through the incision or port.

The embodiment of the retractor 400 illustrated in FIG. 26A is generally linear between the base 402 and the distal portion 410. FIG. 26B illustrates another embodiment that includes a bend 418 between the base 402 and the distal portion 410. The bend 418 may be an angle α. The angle α can be, for example, about 90 degrees, although other angles larger and smaller are also possible.

FIG. 27A illustrates an embodiment a proximal portion 511 of a robotically-actuated medical retractor 500 that includes a connector 520 at a distal end thereof. The connector 520 can be configured to selectively attach to a distal portion 510 of the medical retractor 500 (as shown in FIGS. 27B and 27C). This configuration can allow various distal portions 510 (for example, different distal portions comprising different shapes for supporting the liver) to be selectively attached to the proximal portion 511 as desired. This configuration can also allow for distal portions 510 to be discarded after use or after a limited number of uses, while the proximal portion 511 (including, for example, the robotically actuated base 502) can be configured for multiple uses.

FIG. 27A illustrates an embodiment of the proximal portion 511 of the retractor 500. As illustrated the proximal portion 511 can include a base 502 and an elongated shaft 508. The base 502 can be configured to attach to a robotic arm or instrument driver as previously described. In many respects, the shaft 508 is similar to the shafts previously described, except that the shaft 508 includes a connector 520 at its distal end. The connector 520 can include any releasable attachment or fastening mechanism, such as mechanical latch, threaded attachment, pin and slot attachment, and/or magnetic attachment. The connector is configured to allow the proximal portion 511 to be connected to different distal portions 510. Two example distal portions 510 are shown in FIGS. 27A and 27B.

FIG. 27B illustrates an embodiment of an articulable distal portion 510A that is configured to connect to the proximal portion 511 of the medical retractor 500 of FIG. 27A. Accordingly, the distal portion 510A includes a connector 522 at its proximal end. The connector 522 can include any releasable attachment or fastening mechanism complementary to the connector 520, such as a mechanical latch, threaded attachment, pin and slot attachment, and/or magnetic attachment. The connector 522 is configured to engage with the connector 520 to secure the distal portion 510A to the proximal portion 511. In the illustrated embodiment, the distal portion 510A is configured to articulate in a manner that can be similar to the distal portion 310 of FIG. 25B. To facilitate articulation of the distal portion 510A, the connectors 520, 522 can be configured to connect pull wires within the proximal portion 511 and the distal portion 510. This can allow the distal portion 510A to articulate between a generally linear configuration for insertion and an articulated configuration for supporting the liver.

FIG. 27C illustrates an embodiment of a non-articulating distal portion 510B for the medical retractor 500 including a connector 522 for connecting to the proximal portion 511 of FIG. 27A. In the illustrated example, the non-articulating distal portion 510B comprises a shape that is configured to allow for insertion into the patient and to support the liver. In the illustrated embodiment, the non-articulating distal portion 510B comprises an open loop or U-shape. In some embodiments, the non-articulating distal portion 510B comprises a retractor that can also be used manually. For example, the distal portion 510B can be a manual retractor that could also be manually operated and/or supported with table mounted or manual retractor systems such as those shown in FIG. 21. Thus, FIG. 27A and 27C illustrate that in some embodiments, commercially available manual retractors can be attached to robotically controllable proximal portions 511 (as shown in FIG. 27A) to allow for robotic control of the manual retractor. A proximal portion 511 that attaches to a manual retractor instrument could additionally reduce cost since the end effector and base are decoupled, and there is reduced tool complexity due to the end effector being non-articulating.

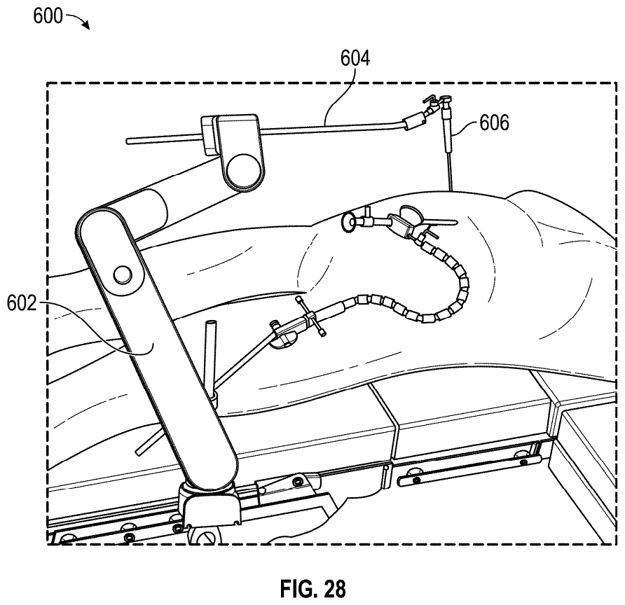

FIG. 28 illustrates an embodiment of a robotic medical system 600 including a robotic arm 602, an instrument 604 comprising articulating segments, and a medical retractor 606. In this embodiment, the instrument 604 can comprise robotically controllable articulating segments or joints such that the shape or pose of the instrument 604 can be robotically controlled. In some embodiments, the instrument 604 comprises one, two, three, four, five or more articulating segments or joints. The instrument 604 can be configured to extend between the robotic arm 602 and the retractor 606 such that the instrument 604 remains positioned outside of the body. However, because the instrument 604 and the robotic arm 602 are robotically controllable, the position of the retractor 606 can be robotically adjusted. The instrument 604 can be used with custom or commercially available retractors. In the system 600, the instrument 604 interfaces with the liver retractor extracorporeally. This may enable a user to maintain the position of the liver retractor in the body while using the robotic arm and articulation sections to avoid collisions with other robotic arms or other objects.

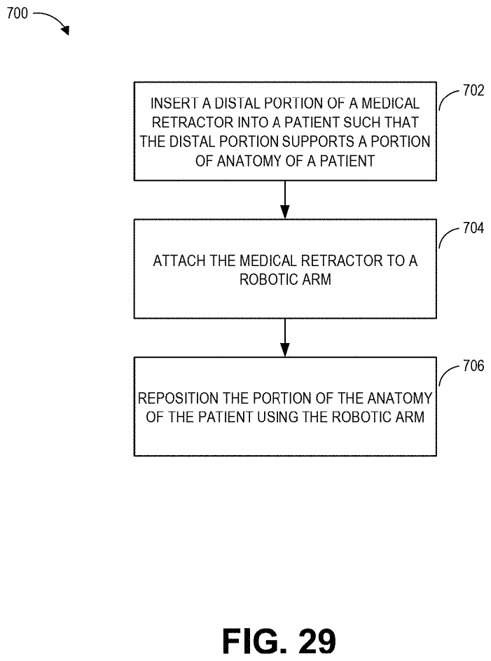

FIG. 29 is a flowchart illustrating an example method 700 for a robotically-enabled medical procedure using a robotically-actuated medical retractor. The method 700 begins at block 702, at which a distal portion of a medical retractor is inserted into a patient such that the distal portion supports a portion of the anatomy of the patient.

At block 704, the medical retractor is attached to a robotic arm. Attaching the retractor to the robotic arm can include attaching a base of the retractor to the robotic arm. In some embodiments, the base of the retractor is attached to an instrument driver on the robotic arm. Instrument drive inputs on the base can engage with corresponding instrument drive outputs on the instrument driver.

In some embodiments, attaching the retractor to the robotic arm occurs prior to inserting the distal portion of the retractor into the patient. This may be the case, for example, where the retractor includes an articulable distal portion that can articulate between a generally linear configuration (e.g., FIG. 22A) and a configuration configured to support a liver (e.g., FIG. 22B). When the retractor is attached to the robotic arm prior to insertion, insertion can be performed robotically using the robotic arm or driven by the instrument driver.

In some embodiments, the retractor is inserted into the patient prior to attaching the retractor to the robotic arm. This may be the case, for example, where the retractor includes a non-articulating distal portion (e.g., FIGS. 26A and 26B). In some instances, when the retractor includes a non-articulating distal portion, the robotic arm may not be capable of sufficient movement to guide the complex rigid distal end into the patient. In such cases, the retractor can be inserted manually and then attached to the robotic arm. Additionally or alternatively, admittance or impedance control of the robotic arm can be used to manually manipulate the retractor for initial insertion or positioning after the retractor has been attached to the arm. The robotic arm can then be manipulated robotically to control the fine positioning of the retractor.

At block 706, the robotic arm is used to reposition the portion of the patient's anatomy. Repositioning can be controlled remotely through the robotic system. This can advantageously allow the physician to adjust the retraction using the robotic system such that adjusting the retraction can be seamlessly performed robotically with the remainder of the procedure. This can reduce total operation time and improve patient outcomes.

In some embodiments, the method 700 further comprises repositioning at least one segment of the robotic arm, while maintaining a position and orientation of the liver retractor, to avoid a collision between the robotic arm and another object. For example, the robotic arm can include one or more redundant degrees of freedom that allow it to be repositioned without adjusting the position of the retractor. This can allow the robotic arm to be repositioned in order to avoid a collision without moving the retractor. In some embodiments, such repositioning can be automatic. For example, the system can automatically reposition a portion of the robotic arm in order to avoid a collision. In some embodiments, the physician commands repositioning of the robotic arm in order to avoid collisions.

In some embodiments, the liver retractor includes an articulable distal portion. In such embodiments, the method 700 can further include robotically articulating the distal portion of the liver retractor with an instrument driver on the robotic arm to adjust the shape or pose of the distal portion.

In some embodiments, the method 700 includes attaching the distal portion of the liver retractor to a proximal portion of the liver retractor. This can be the case where the retractor comprises separate components, such as shown, for example, in FIGS. 27A-27C. Similarly, the method 700 can include detaching the distal portion of the liver retractor from a proximal portion of the liver retractor, and attaching another distal portion to the proximal portion. In some embodiments, the method 700 can include disposing the detached distal portion.

The method 700 can include inserting a laparoscopic tool attached to another robotic arm into the patient, and performing a medical procedure with the laparoscopic tool while the distal portion of the liver retractor supports the liver. In some embodiments, the robotic liver retractor is remotely controlled.

### 3. Implementing Systems and Terminology

Implementations disclosed herein provide systems, methods and apparatus for robotically-actuated medical retractors, such as robotically-actuated liver retractors.

It should be noted that the terms “couple,” “coupling,” “coupled” or other variations of the word couple as used herein may indicate either an indirect connection or a direct connection. For example, if a first component is “coupled” to a second component, the first component may be either indirectly connected to the second component via another component or directly connected to the second component.

The phrases referencing specific computer-implemented processes and/or functions described herein may be stored as one or more instructions on a processor-readable or computer-readable medium. The term “computer-readable medium” refers to any available medium that can be accessed by a computer or processor. By way of example, and not limitation, such a medium may comprise random access memory (RAM), read-only memory (ROM), electrically erasable programmable read-only memory (EEPROM), flash memory, compact disc read-only memory (CD-ROM) or other optical disk storage, magnetic disk storage or other magnetic storage devices, or any other medium that can be used to store desired program code in the form of instructions or data structures and that can be accessed by a computer. It should be noted that a computer-readable medium may be tangible and non-transitory. As used herein, the term “code” may refer to software, instructions, code or data that is/are executable by a computing device or processor.

The methods disclosed herein comprise one or more steps or actions for achieving the described method. The method steps and/or actions may be interchanged with one another without departing from the scope of the claims. In other words, unless a specific order of steps or actions is required for proper operation of the method that is being described, the order and/or use of specific steps and/or actions may be modified without departing from the scope of the claims.

As used herein, the term “plurality” denotes two or more. For example, a plurality of components indicates two or more components. The term “determining” encompasses a wide variety of actions and, therefore, “determining” can include calculating, computing, processing, deriving, investigating, looking up (e.g., looking up in a table, a database or another data structure), ascertaining and the like. Also, “determining” can include receiving (e.g., receiving information), accessing (e.g., accessing data in a memory) and the like. Also, “determining” can include resolving, selecting, choosing, establishing and the like.

The phrase “based on” does not mean “based only on,” unless expressly specified otherwise. In other words, the phrase “based on” describes both “based only on” and “based at least on.”

The previous description of the disclosed implementations is provided to enable any person skilled in the art to make or use the present invention. Various modifications to these implementations will be readily apparent to those skilled in the art, and the generic principles defined herein may be applied to other implementations without departing from the scope of the invention. For example, it will be appreciated that one of ordinary skill in the art will be able to employ a number corresponding alternative and equivalent structural details, such as equivalent ways of fastening, mounting, coupling, or engaging tool components, equivalent mechanisms for producing particular actuation motions, and equivalent mechanisms for delivering electrical energy. Thus, the present invention is not intended to be limited to the implementations shown herein but is to be accorded the widest scope consistent with the principles and novel features disclosed herein.

1-15. (canceled)

16. A medical instrument comprising:
an instrument base configured to attach to an instrument driver of a robotic arm, the instrument base comprising at least one instrument drive input configured to engage a corresponding at least one instrument drive output of the instrument driver;
an elongated shaft extending from the instrument base, the elongated shaft having a distal portion that is configured to articulate between a substantially linear configuration for insertion into a patient and a bent configuration for atraumatically supporting a liver during a robotic medical procedure; and
at least one pull wire positioned between the instrument drive input of the instrument base and the distal portion of the elongated shaft, the pull wire configured to articulate the distal portion.

17. The medical instrument of claim 16, wherein the distal portion comprises:
a first articulating section;
a second articulating section;
a first rigid section positioned between the first articulating section and the second articulating section; and
a second rigid section positioned distally of the second articulating section.

18. The medical instrument of claim 17, further comprising:
a third articulating section positioned distally of the second rigid section; and
a third rigid section positioned distally of the third articulating section.

19. The medical instrument of claim 16, wherein, in the bent configuration, the articulable distal portion comprises a generally triangular shape.

20. The medical instrument of claim 18, wherein:
the at least one instrument drive input comprises a first instrument drive input, a second instrument drive input, and a third instrument drive input; and
the at least one pull wire comprises a first pull wire extending between the first articulating section and the first instrument drive input, a second pull wire extending between the second articulating section and the second instrument drive input, and a third pull wire extending between the third articulating section and the third instrument drive input.

21. The medical instrument of claim 18, wherein the first, second, and third articulating sections are configured to be independently articulable.

22. The medical instrument of claim 16, wherein, in the substantially linear configuration, the distal portion is configured for insertion into the patient via an incision.

23. A robotic medical system comprising:
a robotic arm; and
a medical instrument comprising:
an instrument handle configured to attach to the robotic arm;
a proximal portion that extends from the instrument handle, and
a distal portion configured to support a liver during a robotic medical procedure.

24. The system of claim 23, wherein the proximal portion comprises at least two substantially straight portions connected at an angle.

25. The system of claim 24, wherein the angle is approximately 90 degrees.

26. The system of any of claims 23, wherein the proximal portion is rigid.

27. The system of any of claims 23, wherein the distal portion comprises an open loop shape.

28. The system of claim 27, wherein the open loop shape comprises a U-shape.

29. The system of claim 27, wherein the distal portion is rigid and non-articulable.

30. The system of claim 23, wherein the distal portion comprises a rigid rod with a rounded outer surface and rounded tip.

31. The system of claim 23, wherein the distal portion is configured to be articulable.

32. The system of claim 31, wherein, in an articulated configuration, the distal portion comprises a closed loop shape.

33. The system of claim 32, wherein the closed loop shape comprises a triangular shape.

34. The system of claim 23, further comprising:
a patient platform for supporting a patient during a medical procedure, wherein the robotic arm is attached to the patient platform.

35. The system of claim 23, wherein the robotic arm is a first robotic arm, the system further comprising:
a second robotic arm; and
a robotically-controlled laparoscopic tool attached to the second robotic arm.

36. The system of claim 35, further comprising a processor configured to:
reposition at least one segment of the first robotic arm, while maintaining a position and orientation of the medical instrument, to avoid a collision between the first robotic arm and the second robotic arm used during a medical procedure.

37. A medical instrument comprising:
an instrument handle configured to attach to a distal end of a robotic arm;
an elongated shaft extending from the instrument handle, the elongated shaft having a distal end;
a first connector positioned at the distal end of the elongated shaft; and
a liver retractor configured to atraumatically support of a liver during a robotic medical procedure, the liver retractor comprising a second connector configured to attach to the first connector of the elongated shaft.

38. The medical instrument of claim 37, wherein the liver retractor comprises:
a rigid proximal portion that extends from the second connector, and
a non-articulating distal portion configured to support the liver during the robotic medical procedure.

39. The medical instrument of claim 38, wherein the non-articulating distal portion comprises a U-shape.

40. The medical instrument of claim 37, wherein the liver retractor comprises an articulable distal portion configured to articulate between a substantially linear configuration and a bent configuration configured to atraumatically support the liver during a robotic medical procedure.

41. The medical instrument of claim 40, wherein the bent configuration comprises an open loop configuration.

42. The medical instrument of claim 40, wherein the bent configuration comprises a closed loop configuration.

43. A medical system comprising:
the medical instrument of claim 37;
an instrument driver at the distal end of the robotic arm, the instrument driver comprising at least one instrument drive output configured to engage a corresponding at least one instrument drive output of the instrument handle;
at least one first pull wire segment extending between the instrument drive input of the instrument handle and the first connector; and
at least one second pull wire segment extending between the second connector and the articulable distal portion of the liver retractor, wherein the at least one second pull wire segment is operable coupled to the at least one first pull wire segment when the first connector is connected to the second connector such that articulation of the articulable distal portion of the liver retractor can be driven by the instrument driver.
